(19) United States
(12) Patent Application Publication
(10) Pub. No.: US 2021/0299064 A1
(43) Pub. Date: Sep. 30, 2021

(54) METHOD OF TREATING PATIENTS WITH LENNOX-GASTAUT SYNDROME

(71) Applicant: ZOGENIX INTERNATIONAL LIMITED, Berkshire (GB)

(72) Inventor: Glenn MORRISON, Half Moon Bay, CA (US)

(73) Assignee: ZOGENIX INTERNATIONAL LIMITED, Berkshire (GB)

(21) Appl. No.: 17/164,403

(22) Filed: Feb. 1, 2021

Related U.S. Application Data
(60) Provisional application No. 62/970,630, filed on Feb. 5, 2020.
(60) Provisional application No. 63/120,952, filed on Dec. 3, 2020.

(51) Int. Cl. A61K 31/135; A61K 45/06; A61K 9/00; A61P 25/08

(52) U.S. Cl. CPC A61K 31/135; A61P 25/08; A61K 9/0053; A61K 45/06

(57) ABSTRACT

A method of treating symptoms of Lennox-Gastaut syndrome in a patient diagnosed with Lennox-Gastaut syndrome, by administering an effective dose of fenfluramine to that patient over a period of time sufficient to reduce or completely eliminate seizures in the patient. The fenfluramine may be administering in an oral liquid formulation on a daily basis of 0.7 mg/kg/day, over a period of weeks until seizures are reduced by 25% or more, 50% or more, 75% or more.

### FIELD OF THE INVENTION

A method of treating patients with Lennox-Gastaut syndrome (LGS) is described whereby the patient is repeatedly treated with fenfluramine and the treatment continued to obtain a desired end point not previously recognized.

### BACKGROUND OF THE INVENTION

This invention relates to the treatment of Lennox-Gastaut syndrome (LGS) syndrome using an amphetamine derivative, specifically fenfluramine

Fenfluramine, i.e. 3-trifluoromethyl-N-ethylamphetamine, is an amphetamine derivative having the structure:

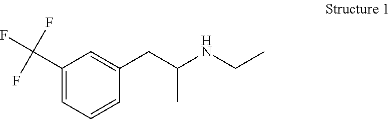 [SMILES: CCNC(C)Cc1cccc(C(F)(F)F)c1]

### (RS)—N-ethyl-1-[3-(trifluoromethyl)phenyl]propan-2-amine

Fenfluramine is often formulated as an acid salt, such as, typically fenfluramine hydrochloride (HCl), while other pharmaceutically acceptable salts may be used. The molecular weight of fenfluramine free base (C₁₂H₁₆F₃N) is 231.26 g/mol and the molecular weight of fenfluramine hydrochloride (C₁₂H₁₆F₃N.HCl) is 267.72 g/mol.

Fenfluramine was first marketed as Pomidin® in the US in 1973 for short term use as an appetite suppressant and its dextrorotatory isomer (d-fenfluramine, Redux®) was approved in 1996 for treatment periods up to one year. It was used “off-label”, i.e., in an unapproved use, by administration in combination with phentermine (“phen/fen”) to treat obesity. Pomidin was supplied as 20 mg tablets and dosed between 40 and 60, and as high as, 120 mg/day. Reports of cardiac valvulopathies and pulmonary hypertension associated with fenfluramine use were published in early 1997 leading to the FDA issuing an advisory on its use in July 1997. Fenfluramine was voluntarily withdrawn from the US market in September 1997 and shortly thereafter in Europe. Subsequently, the drug was withdrawn from sale globally and no longer indicated for use in any therapeutic area. Li, R., et al. (Intl. J. Obesity (1999) 23, 926-928) published an evaluation of severe valvulopathies in obese patients taking 40 mg fenfluramine per day compared with those taking 60 mg (or more) fenfluramine per day and calculated an adjusted odds ratio of 9.2 (95% C.I. 2.1-40.8). From this study and analysis, they concluded that the risk of clinically severe valvular heart disease increased markedly with dose. Low dose fenfluramine for use in treatment of Dravet syndrome is the subject of marketing approval applications submitted in 2019 at the US FDA and European MAA for distribution under a cardio-pulmonary safety monitoring system.

Despite the health concerns surrounding fenfluramine, attempts have been made to identify further therapeutic uses for that product. Aicardi and Gastaut (New England Journal of Medicine (1985), 313:1419 and Archives of Neurology (1988) 45:923-925) reported four cases of self-induced photosensitive seizures that responded to treatment with fenfluramine.

Clemens, in Epilepsy Research (1988) 2:340-343 reported a study on a boy suffering pattern sensitivity-induced seizures that were resistant to anticonvulsive treatment. Fenfluramine reportedly successfully terminated these self-induced seizures and the author concluded that this was because fenfluramine blocked the photosensitive triggering mechanism.

In Neuropaediatrics, (1996); 27(4):171-173, Boel and Casaer reported on a study on the effects of fenfluramine on children with refractory epilepsy. They concluded that when fenfluramine was administered at a dose of 0.5 to 1 mg/kg/day, this resulted in a reduction in the number of seizures experienced by the patients.

In a letter to Epilepsia, published in that journal (Epilepsia, 43(2):205-206, 2002), Boel and Casaer commented that fenfluramine appeared to be of therapeutic benefit in patients with intractable epilepsy.

A JAMA Neurology publication of December 2019 reports the results of a trial of fenfluramine when administered in conjunction with other antiepileptic drugs in the treatment of Dravet syndrome, a refractory epilepsy of childhood that is strongly linked to mutations in the SCN1A gene that codes for the sodium channel subtype 1. Approximately 80% of patients diagnosed with Dravet syndrome have a mutation in SCN1A. U.S. Pat. No. 10,478,442 describes and claims methods of adjunctive treatment of Dravet syndrome with fenfluramine Dravet syndrome is a severe childhood epilepsy syndrome associated with treatment refractory seizures and cognitive disabilities.

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited or de novo genetic mutations, degenerative disease, head injury or trauma, metabolic disorders, cerebrovascular accident and alcohol withdrawal.

Epilepsies may be organized (by reliably identified common clinical and electroencephalographic (EEG) characteristics) into epilepsy syndromes. Such syndromes have a typical age of seizure onset, specific seizure types and EEG characteristics and often other features which when taken together allow the specific epilepsy syndrome diagnosis. The identification of an epilepsy syndrome is useful as it provides information on which underlying etiologies should be considered and which anti-seizure medication(s) might be most useful. Several epilepsy syndromes demonstrate seizure aggravation with particular anti-seizure medications, which can be avoided through appropriate early diagnosis.

There are many subtypes of epilepsy that have been characterized. For example, the most recent classification system adopted by the International League Against Epilepsy's (“ILAE”) Commission on Classification and Terminology provides the following list of epilepsy syndromes (See Berg et. al., “Revised terminology and concepts for organization of seizures,” Epilepsia, 51(4):676-685 (2010)):

I. Electroclinical syndromes arranged by age at onset:

A. Neonatal period (1. Benign familial neonatal epilepsy (BFNE), 2. Early myoclonic encephalopathy (EME), 3. Ohtahara syndrome),

B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Dravet syndrome, 7. Myoclonic encephalopathy in nonprogressive disorders),

C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), 11. Childhood absence epilepsy (CAE));

D. Adolescence—Adult (1. Juvenile absence epilepsy (JAE), 2. Juvenile myoclonic epilepsy (JME), 3 Epilepsy with generalized tonic—clonic seizures alone, 4. Progressive myoclonus epilepsies (PME), 5. Autosomal dominant epilepsy with auditory features (ADEAF), 6. Other familial temporal lobe epilepsies,

E. Less specific age relationship (1. Familial focal epilepsy with variable foci (childhood to adult), 2. Reflex epilepsies);

II. Distinctive constellations: A. Mesial temporal lobe epilepsy with hippocampal sclerosis (MTLE with HS), B. Rasmussen syndrome, C. Gelastic seizures with hypothalamic hamartoma, D. Hemiconvulsion—hemiplegia—epilepsy, E. Other epilepsies, distinguished by 1. presumed cause (presence or absence of a known structural or metabolic condition, then 2. primary mode of seizure onset (generalized vs. focal);

III. Epilepsies attributed to and organized by structural-metabolic causes: A. Malformations of cortical development (hemimegalencephaly, heterotopias, etc.), B. Neurocutaneous syndromes (tuberous sclerosis complex, Sturge-Weber, etc.), C. Tumor, D. Infection, E. Trauma;

IV. Angioma: A. Perinatal insults, B. Stroke, C. Other causes;

V. Epilepsies of unknown cause;

VI Conditions with epileptic seizures that are traditionally not diagnosed as a form of epilepsy per se; A. Benign neonatal seizures (BNS); and B. Febrile seizures (FS).

See Berg et. al, “Revised terminology and concepts for organization of seizures,” Epilepsia, 51(4):676-685 (2010))

As can be seen from, for example, Part V of that list, there are still subtypes of epilepsy that have not yet been fully characterized and thus, the list is far from complete.

Those skilled in the art will recognize that these subtypes of epilepsy are triggered by different stimuli, are controlled by different biological pathways and have different causes, whether genetic, pathologic or environmental. In other words, the skilled artisan will recognize that teachings relating to one epileptic subtype are not necessarily applicable to other subtypes. This can include recognition that different epilepsy subtypes respond differently to different anticonvulsant drugs.

Lennox-Gastaut syndrome (LGS), also known as Lennox syndrome, is a refractory (difficult-to-treat) form of childhood-onset epilepsy that most often appears between the second and sixth year of life and is characterized by frequent seizures and different seizure types; it is often accompanied by developmental delay and psychological and behavioral problems.

An epilepsy syndrome has been defined as a complex of signs and symptoms that defines a unique epilepsy condition [International League Against Epilepsy Task Force on Classification and Terminology]. The classic LGS triad, when the syndrome is fully developed, comprises many types of seizures that include tonic seizures, mental retardation, and an interictal EEG pattern of diffuse, slow spike-wave complexes. Some epileptologists consider the presence of fast (10 Hz) rhythms (bilateral paroxysmal fast activity) that are associated with the tonic attacks (or may occur with minimal manifestations), particularly during non-REM sleep, to be an essential criterion as well. The diagnosis of LGS is dependent on the interaction between the clinical features and the EEG features. [Arzimanoglou, A., et al., Lancet Neurol 2009; 8: 82-93]

Lennox Gastaut syndrome, unlike Dravet syndrome, is not associated with mutations in a specific gene, although there may be genetic predispositions to developing the syndrome. Pathologies that may lead to LGS include early life anoxia, encephalitis, cranial hemorrhage and focal cortical malformations (dysplasias). West syndrome (infantile spasms) can evolve into LGS, but a substantial percentage of such patients who develop LGS have had hemorrhages or anoxic episodes that result from the infantile spasms. [Blume, W. T. Pathogenesis of Lennox-Gastaut syndrome: Considerations and hypotheses. Epileptic Disorders. 2002; 3(4):183-96]

Tuberous sclerosis complex, a genetic disease caused by mutations of either the TSC1 or TSC2 genes, result in uncontrolled growth of tissues and resulting tubers (benign tumors) including in the brain. Such lesions may be associated with the LGS phenotype, however, defects in this molecular pathway, and at times the same genetic abnormality, can produce epileptic spasms, an LGS phenotype, or focal epilepsy. Hence, there are factors other than the specific molecular mechanism that determine whether a patient will express the LGS phenotype. [Archer, J. S., et al. Front Neurol. 2014; 5: 225].

Lennox-Gastaut syndrome may also be classified as cryptogenic, meaning the cause is unknown or cannot be determined after evaluation. Cryptogenic cases are presumed to result from an unidentified condition (secondary Lennox-Gastaut syndrome). Individuals with cryptogenic Lennox-Gastaut syndrome do not have a previous history of seizure activity, prior neurological problems or cognitive impairment before the development of the disorder. Cryptogenic cases generally have a later onset than symptomatic cases.

Although no single pathophysiology underlies the development of LGS, the age-dependent expression implies that there is something about the immature brain that renders it susceptible to development of the LGS phenotype. When this susceptibility coincides with excitatory conditions and/or genetically-induced aberrant cortical development or physiology, an enduring epileptogenic system develops causing chronic intractable epilepsy. [Blume, et al. op. cit.]

LGS was named for neurologists William G. Lennox (Boston, USA) and Henri Gastaut (Marseille, France). In 2010 the first documentary highlighting this disorder was produced by Eisai Inc. in cooperation with the LGS Foundation.

The age of onset of seizures is typically between 2 and 6 years old, though onset can occur at an earlier or later age. The overall prevalence and incidence of LGS is quite low, about 1% of childhood epilepsies and 0.5/100,000/year among children <10 years of age, respectively.

Daily multiple seizures of different types are typical in LGS. Also typical is the broad range of seizures that can occur, larger than that of any other epileptic syndrome. The most common seizure types are tonic-axial, atonic, and atypical absence seizures, but generalized tonic-clonic, myoclonic and partial seizures can also occur in any LGS patient.

Atonic, atypical absence, tonic, complex partial, focalized and tonic-clonic seizures are also common. Additionally, many LGS patients will have status epilepticus, often of the nonconvulsive type, which is characterized by dizziness, apathy, and unresponsiveness. In addition, most patients have atonic seizures, also called drop seizures, which cause their muscles to go limp and result in the patient suddenly and unexpectedly to fall to the ground, often causing significant injury, which is why patients often wear a helmet to prevent head injury. Tonic seizures may also cause drop seizures.

In addition to daily multiple seizures of various types, children with LGS frequently have arrested/slowed psycho-motor development and behavior disorders.

Valproate or divalproex sodium (Depakene®, Depakote®) is generally considered the first-line therapy for Lennox-Gastaut syndrome because it is effective against a wide spectrum of seizures. Valproate is usually first given alone (monotherapy) and if ineffective, another drug such as lamotrigine, topiramate, rufinamide or clobazam may be added. The valproates (acid and salts) represent older AEDs and so were not approved specifically to treat LGS. Currently, there are seven USFDA approved anti-epileptic drugs (AEDs) that are indicated for treating LGS: felbamate (GABA_{A} positive modulator and NMDA receptor blocker), topiramate (multiple mechanisms), lamotrigine (Nat channel blocker), rufinamide (prolongation of inactive state of voltage-gated sodium channels), clonazepam (benzodiazepine GABA_{A} agonist), clobazam (benzodiazepine GABA_{A} agonist), and cannabidiol (Epidiolex®).

Several properties of topiramate likely contribute to its therapeutic effects; it stimulates GABA_{A} receptor activity at brain non-benzodiazepine receptor sites and reduces glutamate activity at both AMPA and kainate receptors. By increasing GABA activity and inhibiting glutamate activity, topiramate may block neuronal excitability, Additionally, it blocks voltage-dependent sodium channels, which may further block seizure activity. Rufinamide modulates the activity of sodium channels and, in particular, prolongation of the inactive state of the channel.

The mechanism of action of cannbidiol in treating seizures is not well understood. CBD may exert a cumulative anti-convulsant effect by modulating a number of endogenous systems including, but not limited to neuronal inhibition (synaptic and extra-synaptic GABA channels), modulation of intracellular calcium (via TRPV, VDAC, GPR55), and possible anti-inflammatory effects (adenosine). CBD does not directly bind to, nor activate, CB1 and CB2 receptors at concentrations pharmacologically relevant to its anticonvulsant effect. Among the likely mechanisms of action, modulation of intra-cellular calcium via GPR-55, TRPV, and VDAC is under active investigation. Additional mechanisms under exploration include adenosine modulation, glycine and GABAergic modulation, and serotonin agonism. (From Epidiolex company website: https://www.gwpharm.com/healthcare-professionals/research/mechanism-action, which was visited Jan. 23, 2020).

Given the suboptimal treatment of seizures in the majority of LGS patients, even with polytherapy, the serious side effects associated with therapies such as felbamate and the developmental and cognitive consequences believed to be caused, at least in part, by frequent childhood seizure activity, there is a medical need for a new anticonvulsant treatment with a novel mechanism of action that can significantly reduce seizure activity in LGS, as well as the need to provide an improved method for treating or preventing the symptoms of Lennox-Gastaut syndrome (LGS) and/or for treating, preventing and/or reducing seizures experienced by sufferers of LGS.

### SUMMARY OF THE INVENTION

A method of treating symptoms of Lennox-Gastaut syndrome in a patient diagnosed with Lennox-Gastaut syndrome, by administering an effective dose of fenfluramine to that patient over a period of time sufficient to reduce or completely eliminate seizures in the patient. The fenfluramine may be administering in an oral liquid formulation on a daily basis of 0.7 mg/kg/day, over a period of weeks until seizures are reduced by 25% or more, 50% or more, 75% or more.

In another aspect of the present invention, there is provided a method of treating, improving or delaying decline in cognition or behavioral measures of communication and social skills as measured by either the BRIEF-S (Behavior Rating Inventory of Executive Function) or VABS (Vineland Adaptive Behavior Scale) assessment tools in a patient diagnosed with Lennox-Gastaut syndrome, by administering an effective dose of fenfluramine to that patient over a period of time sufficient to treating, improving or delaying decline in cognition or behavioral measures of communication and social skills.

In some aspects, the fenfluramine is administered as fenfluramine hydrochloride. In other aspects, the fenfluramine is administered as fenfluramine free base.

In some aspects, the effective dose is administered in the form of one or more dosage forms for oral, injectable, transdermal, inhaled, nasal, rectal, vaginal or parenteral delivery.

In some aspects, the effective dose is administered as an oral solution.

In some aspects, the effective dose ranges from 1.5 mg/kg/day to 0.05 mg/kg/day.

In some aspects, the fenfluramine or pharmaceutically acceptable salt thereof is co-administered with one or more co-therapeutic agents. In some aspects, the co-therapeutic agent comprises an anticonvulsant.

### BRIEF DESCRIPTION OF THE DRAWINGS

FIG. 1 is a patient questionnaire consisting of 20 pages.

FIG. 2 is a flow diagram that schematically shows the study design of the prospective randomized, double-blind, placebo-controlled study of FINTEPLA in children and adults with Lennox Gastaut syndrome. Dosage quantities are calculated for fenfluramine free base.

FIG. 3 is a chart showing the balance of demographics in both treatment arms and the placebo arm of the study.

FIG. 4 is a chart that summarizes the percentage reduction in the primary endpoint: the change in frequency of the mean monthly drop seizures: FINTEPLA 0.7 mg/kg/day compared to placebo.

FIG. 5 is a chart showing the percentage of patients who achieved ³25%, ³50% and ³75% at a maintenance dose of 0.7 mg/kg/day compare with placebo.

FIGS. 6A and 6B show the proportion of patients who showed improvements in the Clinical Global Impression—Improvement (CGI-I) scale as ranked by the principal investigator, grouped as any improvement (minimal, much and very much improved) as shown in 6A and much and very much improved as shown in 6B.

FIGS. 7A and 7B show a percentage of patients who achieved ³25%, ³50% and ³75% at a maintenance dose of 0.7 mg/kg/day compare with placebo calculated. In FIG. 7A the showing is for the Titration+Maintenance Period (weeks 0 to 14) and FIG. 7B the showing is for Maintenance Period only (MPO) (weeks 3-14) showing a trend toward higher response rates in the MPO analysis.

FIG. 8 is a chart showing median reduction in monthly drop seizures for each treatment arm.

FIG. 9 is a table summarizing treatment emergent adverse events for each arm of the study.

FIG. 10 is a schematic representation of numbers of subjects enrolled in each arm of the study and patients who discontinued treatment and numbers of patients who completed the study.

FIG. 11 shows the percent difference from placebo in reduction of monthly drop seizures in (dosages shown for fenfluramine hydrochloride.

FIG. 12 shows the median percent change from baseline for Titration+Maintenance showing a greater response during the MPO at both doses.

FIG. 13 shows the proportion of patients who achieved ³25%, ³50% and ³75% reductions during the maintenance period (MPO) and the Titration+Maintenance Period.

FIG. 14 shows the screening and allocation of subjects into various testing groups.

FIG. 15 shows demographic information about the subjects.

FIG. 16 shows primary efficacy endpoints.

FIG. 17 shows primary efficacy endpoints: estimated median difference from placebo (Hodges-Lehmann Estimate).

FIG. 18 shows responder analysis.

FIG. 19 consists of FIGS. 19A and 19B which shows the median percentage reduction from baseline for patients with generalized tonic-clonic (GTC) and tonic-atonic seizures.

FIG. 20 consists of FIGS. 20C and 20D which shows the median percentage reduction from baseline for patients with tonic seizures and atonic seizures.

FIG. 21 shows the effect of administering fenfluramine on seizures associated with a drop relative to other recently reported antiepileptic drugs (AEDS). P values are relative to placebo. AEDs, antiepileptic drugs; CBD, cannabidiol; fenfluramine, fenfluramine; LGS, Lennox-Gastaut syndrome; NS, not statistically significant; RCTs, randomized controlled trials.

Dosage quantities in all the above figures are provided for fenfluramine as the free base, unless otherwise noted.

### DETAILED DESCRIPTION OF THE INVENTION

Before the present methods of treatment are described, it is to be understood that this invention is not limited to particular method described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a step of administering” includes a plurality of such steps and reference to “the symptom” includes reference to one or more symptoms and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “fenfluramine” and “fenfluramine free base” are used interchangeably herein to refer to a compound with the structure (RS)—N-ethyl-1-[3-(trifluoromethyl)phenyl]propan-2-amine and the Chemical Abstract Services (CAS) number 458-24-2.

The term “pharmaceutically acceptable salt of fenfluramine” includes, but is not limited to, fenfluramine hydrochloride.

The term “reduction from baseline” is used throughout in order to refer to a reduction relative to the same or similar patient prior to administration of fenfluramine During the baseline period, the patient is treated with other therapeutic agents, except for fenfluramine Treatment with the same other therapeutic agents is substantially maintained during the treatment with fenfluramine. The comparison is made relative to the baseline period.

The term “treatment” and “treating” refer to obtaining a desired pharmacological and/or physiological effect. “Treating symptoms of Lennox-Gastaut syndrome” refers to the total or partial prevention of seizures. Treatment includes preventing seizures for a period of time larger than the period between seizures experienced by a patient in the absence of treatment. Ideally, the methods of the present invention result in a total prevention of seizures. However, the invention also encompasses methods in which the instances of seizures are decreased by at least 50%, at least 60%, at least 70%, at least 80% or at least 90%, or periods of seizure freedom are increased by days, weeks, months or years.

The units “mg/kg/day” refers to how many milligrams of a compound are administered per kilogram of body mass of the patient per day. For example, for a dosage of 0.5 mg/kg/day of fenfluramine, a patient weighing 40 kg would receive 20 mg of fenfluramine each day. For a dosage of 0.5 mg/kg/day of fenfluramine hydrochloride, a patient weighting 40 kg would receive 20 mg of fenfluramine hydrochloride each day, which corresponds approximately to 17.3 mg of fenfluramine free base.

### Specific Embodiments

After extensive research, it has unexpectedly been found that fenfluramine can be used to treat, or at least minimize the effects of Lennox Syndrome.

Fenfluramine has been known to trigger the release of serotonin and inhibit serotonin reuptake in the brain due to disruption of its vesicular storage. However, until the present invention was made, it was not known that fenfluramine's mechanism of action made it suitable for the treatment of Lennox Gastaut Syndrome (LGS). Fenfluramine presents a novel mechanism of action among approved antiepileptic drugs.

Thus, according to a still further aspect of the present invention, there is provided a method of stimulating one or more 5-HT receptors in the brain of a patient by administering an effective dose of fenfluramine to said patient, said one or more 5-HT receptors being selected from one or more of 5-HT1A, 5-HT1B, 5-HT1C, 5-HT1D, 5-HT1E, 5-HT1F, 5-HT2A, 5-HT2B, 5-HT2C, 5-HT3, 5-HT4, 5-HT5, 5-HT5A, 5-HT5B 5-HT6, and 5-HT7 amongst others. In certain embodiments of this aspect of the invention, the patient has been diagnosed with Lennox-Gastaut syndrome. In another aspect, fenfluramine acts as a serotonin releasing agent and thus as an indirect 5-HT receptor agonist.

In embodiments of the invention, any effective dose of fenfluramine can be employed. However, surprisingly low doses of fenfluramine have been found by the inventors to be efficacious, particularly for inhibiting, preventing or eliminating seizures in Lennox-Gastaut syndrome patients. Thus, in preferred embodiments of the invention, a daily dose of fenfluramine hydrochloride is less than about 2.0 mg/kg/day, about 1.5 mg/kg/day, about 1.4 mg/kg/day, about 1.3 mg/kg/day, about 1.2 mg/kg/day, about 1.1 mg/kg/day, about 1.0 mg/kg/day, about 0.9 mg/kg/day, about 0.8 mg/kg/day, about 0.7 mg/kg/day, about 0.6 mg/kg/day, about 0.5 mg/kg/day, about 0.4 mg/kg/day, about 0.3 mg/kg/day, about 0.2 mg/kg/day to about 0.1 mg/kg/day, or about 0.05 mg/kg/day. Put differently, a preferred dose is less than about 1.0 mg/kg/day to about 0.05 mg/kg/day. Such a dose is less than the daily dose of fenfluramine hydrochloride suggested for administration to achieve weight loss. Conversion to doses of free base fenfluramine or another acid salt can be effected by dividing the particular dose by the molar weight in g/mol of fenfluramine hydrochloride and then multiplying by the molecular weight of fenfluramine or a fenfluramine salt. For example, 0.2 mg of fenfluramine HCl divided by 267.72 and then multiplied by 231.26 gives 0.173 mg as a corresponding dose of fenfluramine free base. For further example, 0.2 mg of fenfluramine HCl divided by 267.72 and then multiplied by 312.17 (molecular weight of fenfluramine hydrobromide) corresponds to a dose of 0.233 mg when administering as the fenfluramine hydrobromide salt.

In some cases, the daily dose of fenfluramine ranges from 2.0 mg/kg/day to 0.05 mg/kg/day, such as from 1.5 mg/kg/day to 0.05 mg/kg/day, 1.0 mg/kg/day to 0.1 mg/kg/day or from 0.85 mg/kg/day to 0.15 mg/kg/day. In some cases, the daily dose is 0.2 mg/kg/day. In other cases, the daily dose is 0.8 mg/kg/day.

In some cases, the fenfluramine is administered as a pharmaceutically acceptable salt, e.g., fenfluramine hydrochloride. In some cases, the administration involves administering an oral solution of fenfluramine hydrochloride.

In some cases, the administration is a twice daily administration of an oral solution of fenfluramine hydrochloride such that the daily dose is 0.8 mg/kg/day. In some cases, the administration is a twice daily administration of an oral solution of fenfluramine hydrochloride such that the daily dose is 0.2 mg/kg/day.

Fenfluramine can be administered once a day, twice a day, three times a day, four times a day, or more times a day. In some cases, fenfluramine is administered twice a day. In some cases, each dose administered throughout the day has the same amount of fenfluramine.

In some cases, the administration is repeated for 20 days or more, such as for 50 days or more, 100 days or more, or 200 days or more.

In some cases, fenfluramine is administered as an adjunctive pharmaceutical compound. In other words, in order to treat Lennox-Gastaut syndrome, the patient receives not only fenfluramine but also one or more other pharmaceutically active ingredients. Such administrations are sometimes referred to as combination therapy or polytherapy. In some cases, the subject has been receiving a non-fenfluramine active pharmaceutical ingredient for treating Lennox-Gastaut syndrome for a period of time before receiving fenfluramine, e.g., for 1 week or more, 10 weeks or more, 20 weeks or more, 50 weeks or more, or 100 weeks or more. In some cases, the other active pharmaceutical ingredient is stiripentol. In some cases, the other active pharmaceutical ingredient is not stiripentol.

In some cases, the patient is male or nonpregnant, nonlactating female, age 2 to 35 years.

As indicated above the dosing is based on the weight of the patient. However, for convenience the dosing amounts may be preset such as in the amount of 1 mg, 2.5 mg, 5 mg, 10 mg, 15 mg, 20 mg, 30 mg, 40 mg, 50 mg. In general the smallest dose which is effective should be used for the particular patient. That dose is generally well below the dosing used in weight loss and as such well below the 20 mg dose amounts administered for weight loss.

The dose of fenfluramine administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

Particular formulations of the invention are in a liquid form. The liquid may be a solution or suspension and may be an oral solution or syrup which is included in a bottle with a pipette which is graduated in terms of milligram amounts which will be obtained in a given volume of solution. The liquid solution makes it possible to adjust the solution for small children which can be administered anywhere from 0.5 mg to 15 mg and any amount between in half milligram increments and thus administered in 0.5, 1.0, 1.5, 2.0 mg, etc.

An aspect of the invention includes a kit for treating and or preventing symptoms of LGS in a patient diagnosed with LGS, the kit comprising:

a container holding a liquid formulation of fenfluramine;

a pipet connected to the container and configured to withdraw the liquid formulation from the container, wherein the pipet is marked with levels of graduation noting volume of formulation withdrawn;

instructions for administering the liquid formulation to a patient in order to treat LGS.

The formulation may be a formulation or suspension and is prepared such that a given volume of the solution contains a known amount of active fenfluramine. For example, the pipet is graduated in one millimeter amounts and the formulation is characterized such that one millimeter in volume of formulation includes precisely one milligram of fenfluramine. In this manner, the patient may be correctly dosed with a desired milligram dosage of fenfluramine based on a milliliter volume of liquid formulation administered to the patient orally.

In one aspect of the invention, the fenfluramine is used to treat or prevent symptoms of LGS by itself, i.e. as a monotherapy without the use of additional drugs.

The dosage form of fenfluramine employed in the methods of the present invention can be prepared by combining fenfluramine with one or more pharmaceutically acceptable diluents, carriers, adjuvants, and the like in a manner known to those skilled in the art of pharmaceutical formulation.

In a method of the present invention, fenfluramine can be employed as a monotherapy in the treatment of Lennox-Gastaut syndrome. Alternatively, fenfluramine can be co-administered simultaneously, sequentially or separately with one or more co-therapeutic agents, such as anticonvulsants. Preferred co-therapeutic agents can be selected from the group consisting of carbamazepine, ethosuximide, fosphenytoin, lamotrigine, levetiracetam, phenobarbitol, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt of a co-therapeutic agent is also contemplated.

Fenfluramine can be administered in the form of the free base, or in the form of a pharmaceutically acceptable salt, for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm Sci. (1977) 68(1): 1-19.

Fenfluramine for use in the methods of the present invention may be produced according to any pharmaceutically acceptable process known to those skilled in the art. Examples of processes for synthesizing fenfluramine are provided in the following documents: GB1413070, GB1413078 and EP441160.

The dose of fenfluramine to be used in a method of the present invention can be provided in the form of a kit, including instructions for using the dose in one or more of the methods of the present invention. In certain embodiments, the kit can additionally comprise a dosage form comprising one or more co-therapeutic agents.

A method of the present invention can be practiced on any appropriately diagnosed patient. In a typical embodiment of the present invention, the patient is aged about 18 or less, about 16 or less, about 14 or less, about 12 or less, about 10 or less, about 8 or less, about 6 or less or about 4 or less to about 0 months or more, about 1 month or more, about 2 months or more, about 4 months or more, about 6 months or more or about 1 year or more. Thus, the diagnosed patient is typically about one month old to about 18 years old when treated.

The patient with Lennox-Gastaut syndrome (LGS) can experience a variety of seizure types, e.g. generalized tonic-clonic, tonic-atonic, tonic, atonic, or a combination thereof. The term “tonic seizure” refers to a seizure involving an increase in muscle tone during the seizure, i.e. an increase in muscle contraction during the seizure. For instance, a patient can experience involuntary muscle contractions during a tonic seizure, e.g. causing a standing patient to fall. “Atonic” seizures refer to seizures with a decrease in muscle tone, i.e. a decrease in muscle contraction, e.g. resulting in a standing patient to fall. “Clonic” refers to rhythmic muscle contractions, i.e. alternating contractions and relaxations of muscles. The seizure can involve any combination of tonic, atonic, and clonic phases. For example, generalized tonic-clonic seizures (GTCs) involve a period of tonic seizure as well as a period of clonic seizure. As another example, tonic-atonic seizures involve a period of tonic seizure and a period of atonic seizure. A seizure can also include two or more periods of each seizure type.

GTCs are a primary risk factor for sudden unexpected death in epilepsy (SUDEP) and are associated with a 10-fold increased risk of SUDEP. [Sveinsson O, et al. Neurology. 2017; 89(2):170-7].

In some cases, the fenfluramine or pharmaceutically acceptable salt thereof is in the form of an oral solution, i.e., an aqueous solution. In such cases the method includes administering the pharmaceutical composition orally.

In some cases, the fenfluramine or pharmaceutically acceptable salt thereof is co-administered with one or more co-therapeutical agents. The term “co-therapeutic agent” is used interchangeably herein with the term “drug”, “pharmaceutical drug”, and “active pharmaceutical ingredient”. Exemplary co-therapeutic agents include anticonvulsants. The term “anticonvulsant” refers to a pharmaceutical drug used to decrease the frequency of convulsions, e.g. reduce the frequency of epileptic seizures. Exemplary anticonvulsants include carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, and midazolam or a pharmaceutically acceptable salt thereof.

Provided is a method of treating symptoms of LGS in a patient diagnosed with LGS comprising administering an effective dose of fenfluramine or a salt thereof to the patient. For instance, the method can include administering fenfluramine hydrochloride, wherein hydrochloride is a pharmaceutically acceptable salt, e.g. it can be administered as part of the pharmaceutical composition without significant, or any, negative health consequences to the patient. In some cases, but not necessarily all cases, the salt of fenfluramine is a pharmaceutically acceptable salt.

The method can include diagnosing the patient with LGS before the administering. The diagnosing can be performed by a healthcare professional, such as a physician, a psychiatrist, a registered nurse, or a psychologist.

Many different types of epilepsy are known to the medical community, wherein Lennox-Gastaut syndrome is one type of epilepsy. It is understood that other types of epilepsy such as Doose syndrome and Dravet syndrome are sometimes associated with symptoms that are similar to the symptoms of LGS. In some cases, the diagnosis step includes determining that the patient does not have Doose syndrome or Dravet syndrome.

Some indications of LGS include the age at which symptoms first appeared, the type or types of seizures, seizure frequency, cognitive function, a clinician's impression of symptoms, electroencephalogram (EEG) measurements, and genetic mutations. Typically, but not always, symptoms such as seizures begin in childhood, such as before the eleventh birthday of the patient, before the eight birthday, or before the fifth birthday. As used herein, a patient's first birthday occurs one calendar year after the birth of the patient. As such, at the first birthday, a patient is approximately 365 days old, and the patient is approximately 1,825 days old on their fifth birthday. Another parameter that can be assessed during the diagnosis step is the type or types of seizures, wherein LGS is sometimes associated with patients who have multiple types of seizures.

The diagnosis can also be based on seizure frequency. For example, the number of seizures in a 10 day time frame can be measured and then divided by 10 get obtain the average number of seizures per day, e.g. the patient has 0.5 or more seizures per day on average, such 1.0 or more, or 2.0 or more.

The diagnosis can involve measuring cognitive function of the patient because LGS is sometimes associated with cognitive dysfunction or abnormally slow cognitive development. Exemplary methods for assessing cognitive function include the Behavior Rating Inventory of Executive Function (BRIEF) assessment and the Vineland Adaptive Behavior Scale (VABS).

Some BRIEF assessments include 86 questions that test the patient's abilities in eight different aspects of executive function: inhibition (e.g. ability to control impulses), shifting attention (e.g. ability to move focus from a first activity or idea to a second activity or idea), emotional control (e.g. ability to regulate emotional response), initiation (e.g. ability to start an activity and independently generate problem solving strategies), working memory (e.g. ability to retain and manipulate multiple pieces of information simultaneously), planning (e.g. ability to set goals), organization (e.g. ability to place physical items in logical and orderly locations), and self-monitoring (e.g. ability to assess one's own progress or performance on a task). The term “e.g.” used herein indicates examples of each aspect of executive function, and does not denote a definition of each aspect of executive function. As such, the diagnosing can include assessing the patient in one or more of the eight aspects of executive mentioned above regarding BRIEF, i.e. inhibition, shifting attention, emotional control, initiation, working memory, planning, organization, and self-monitoring.

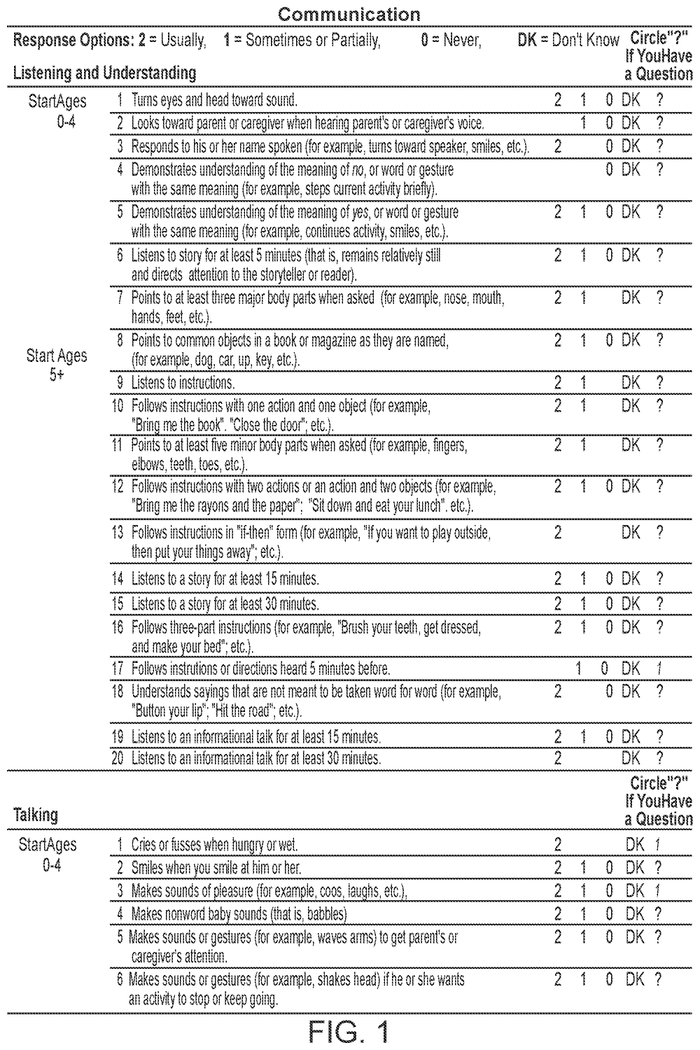

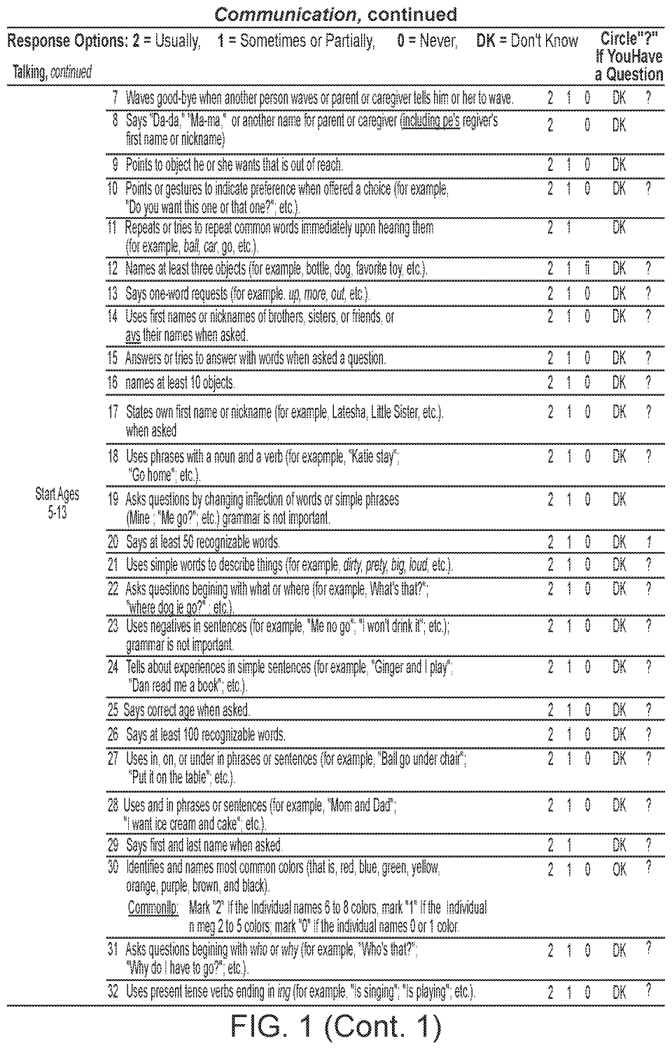

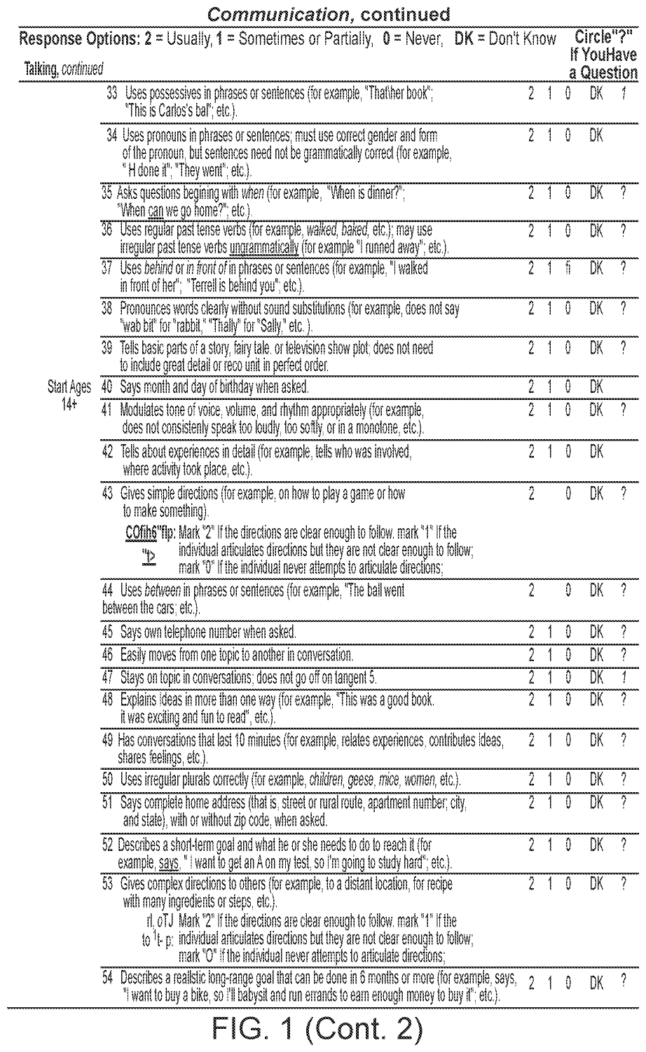

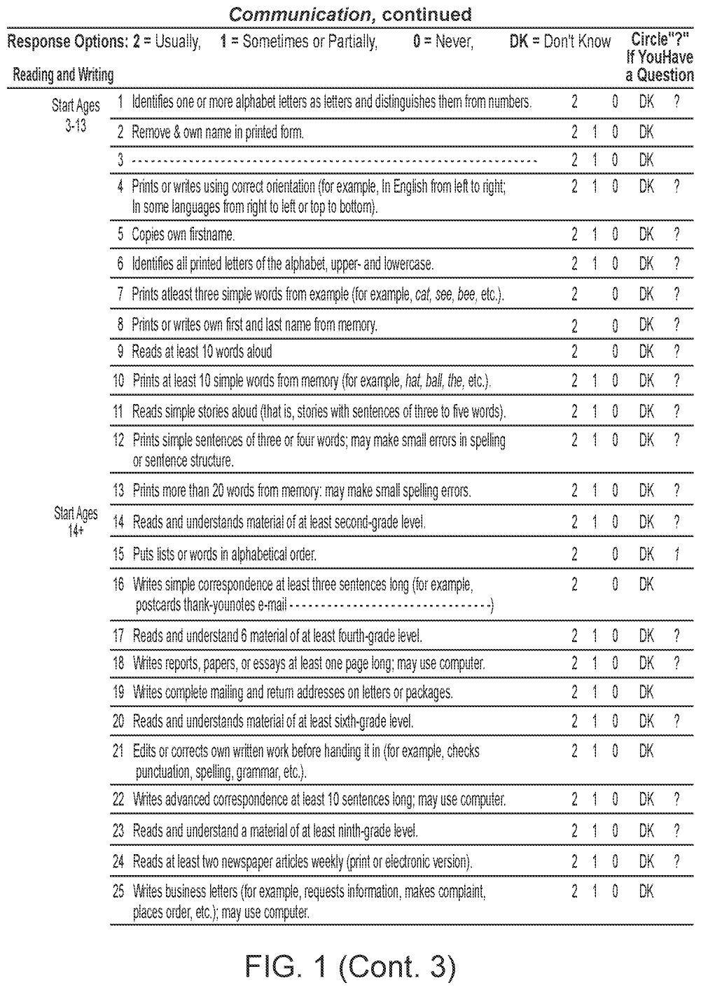

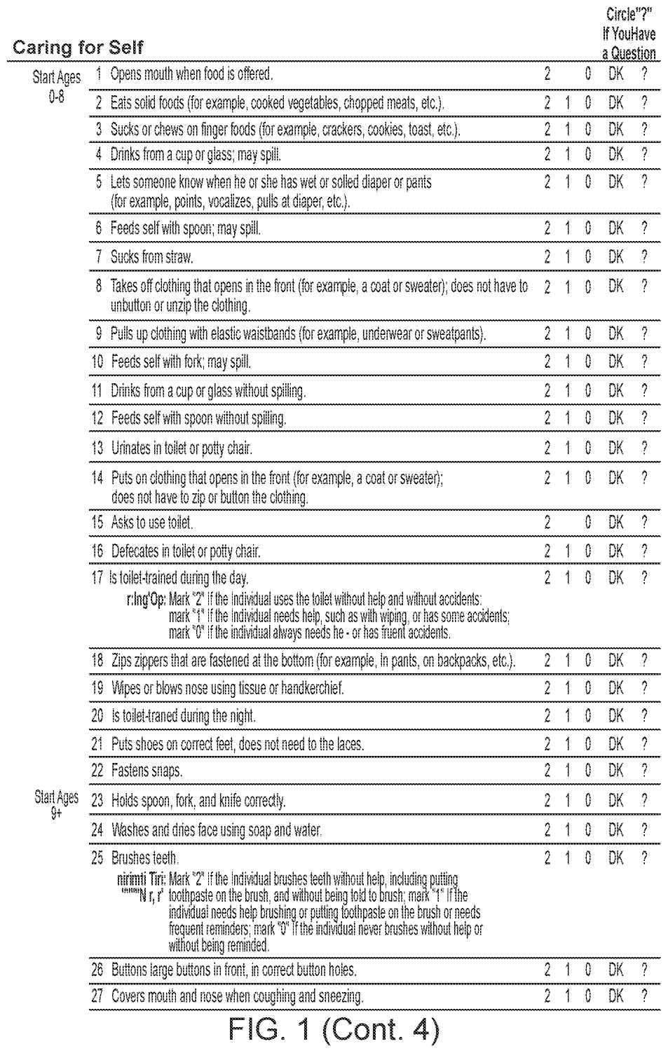

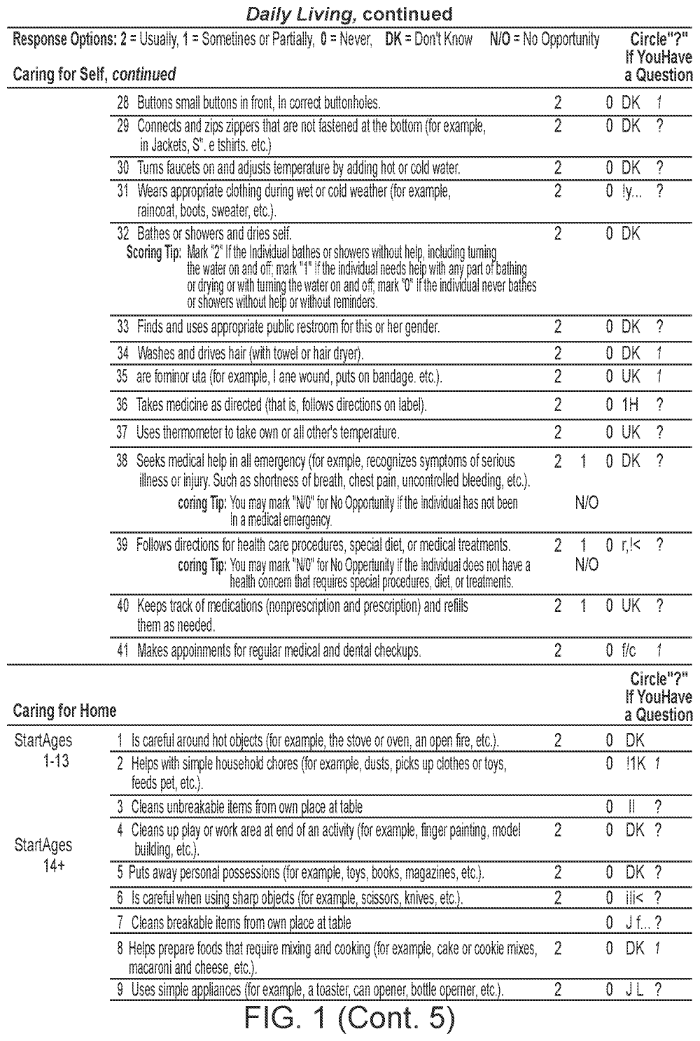

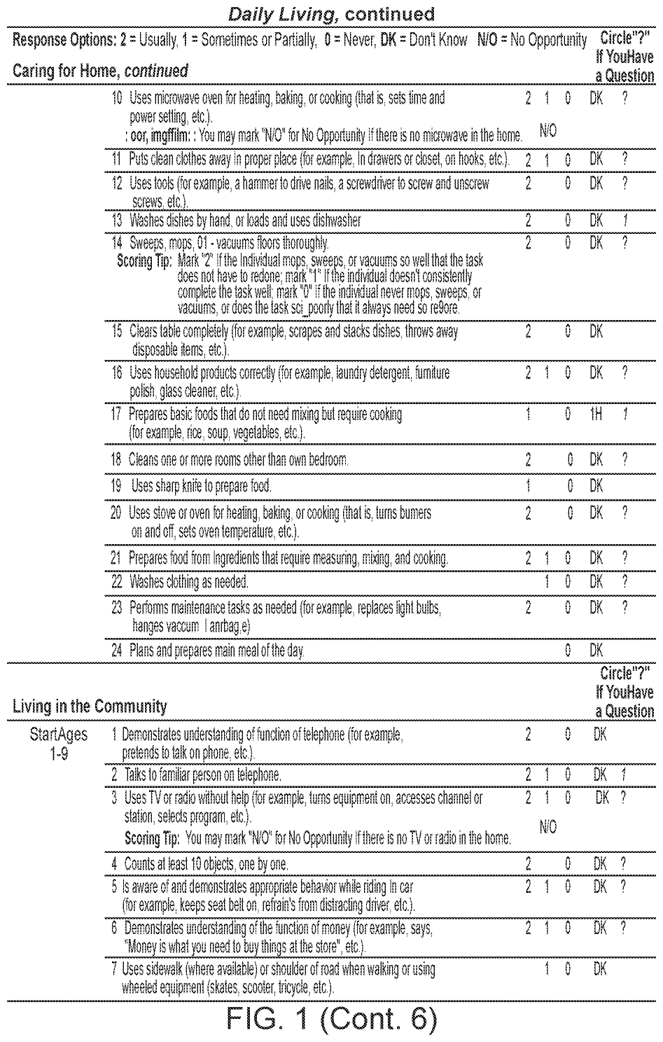

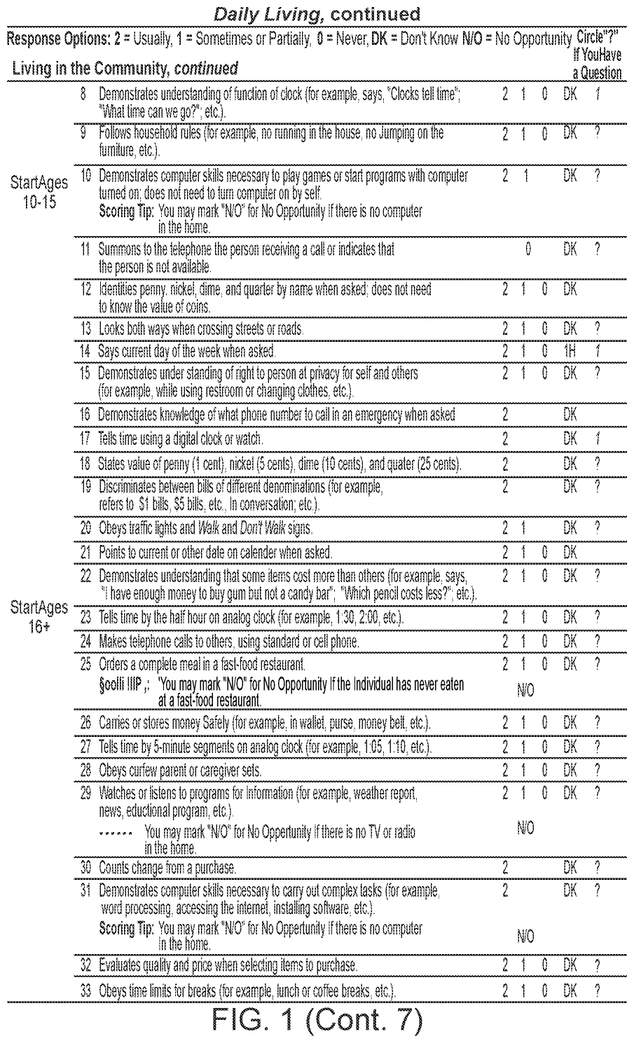

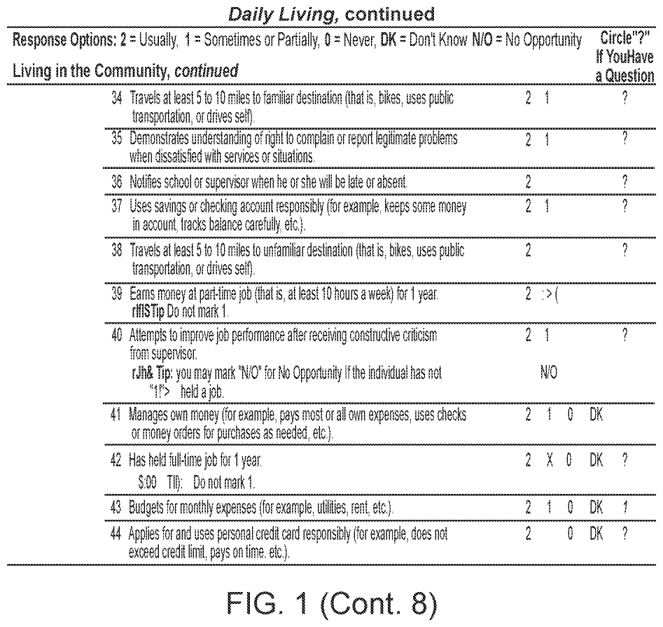

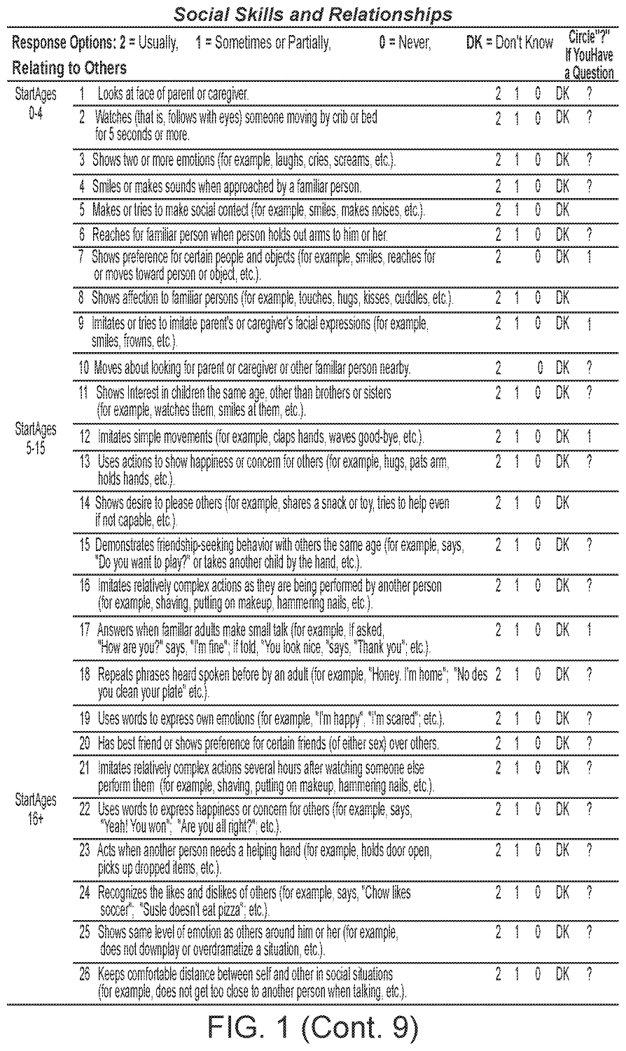

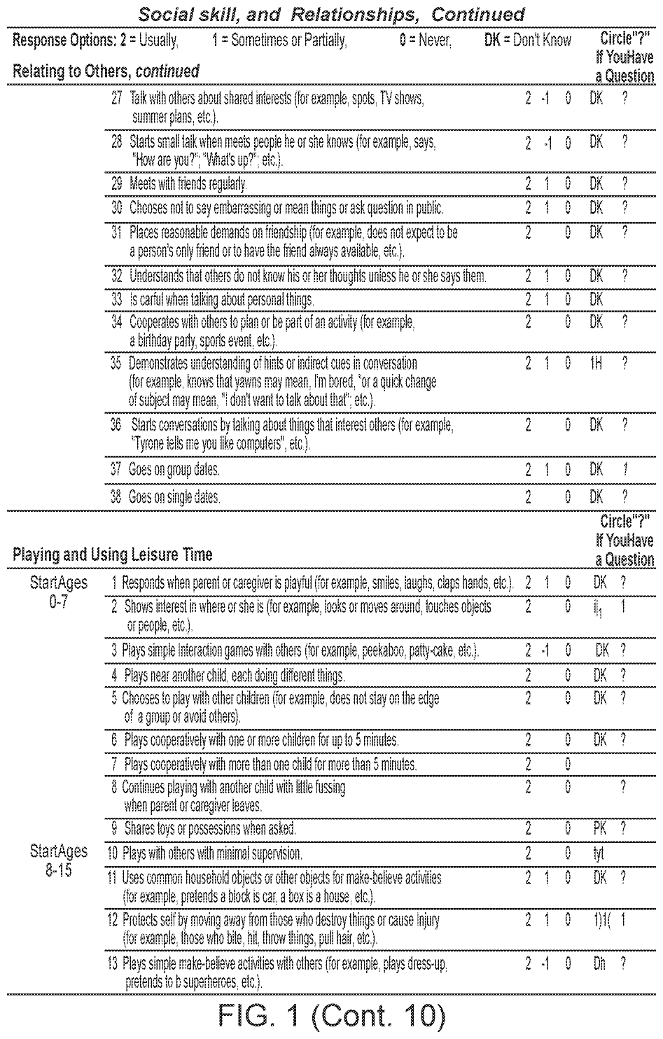

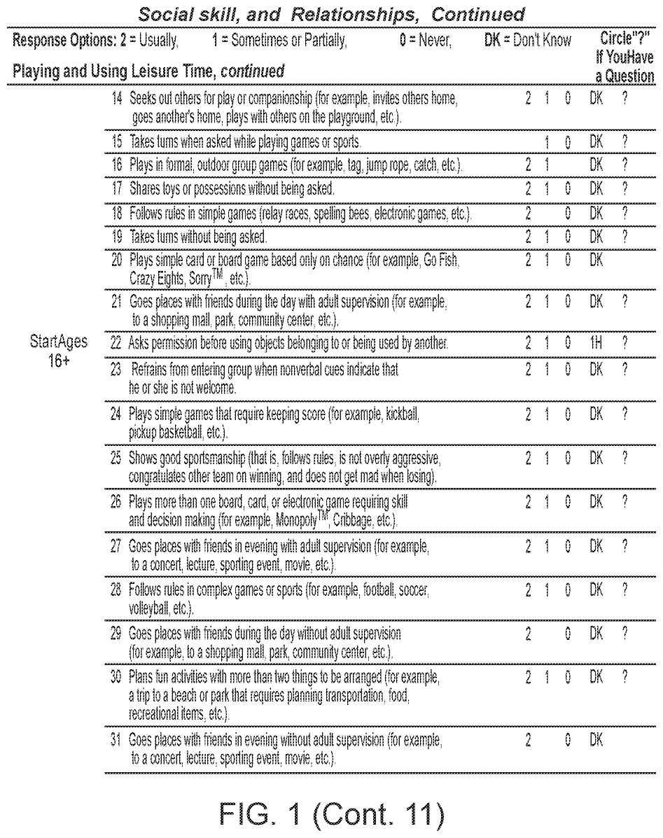

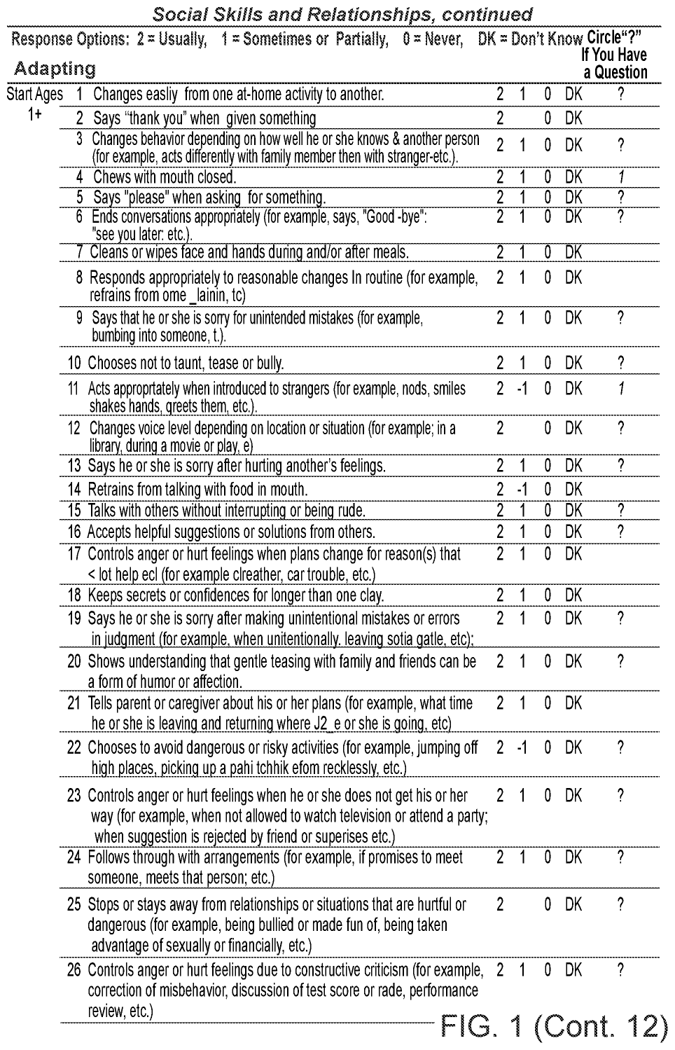

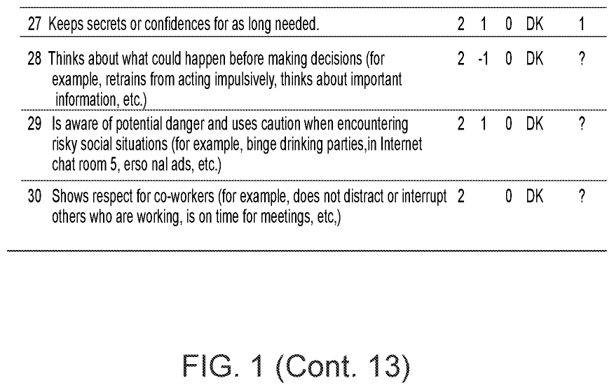

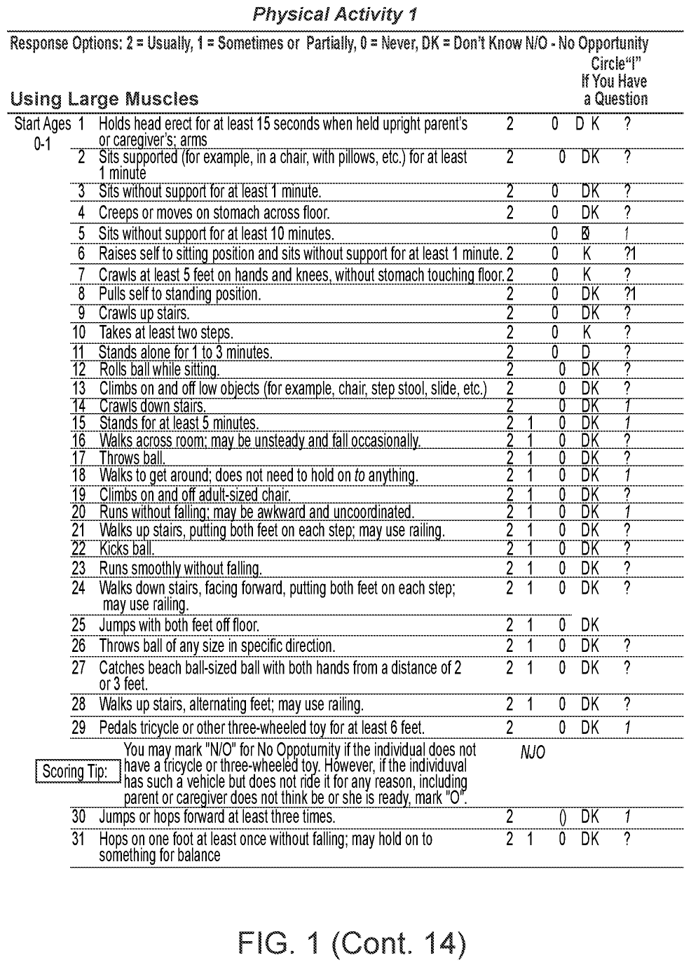

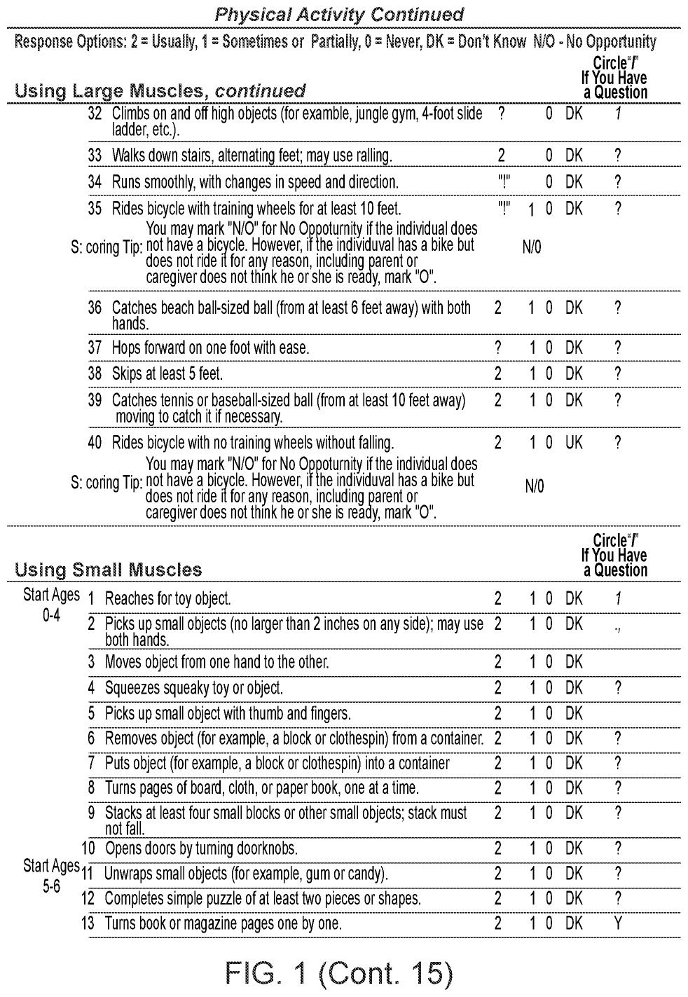

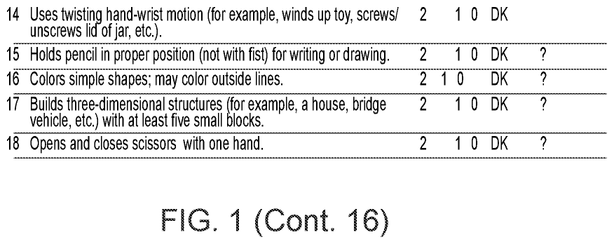

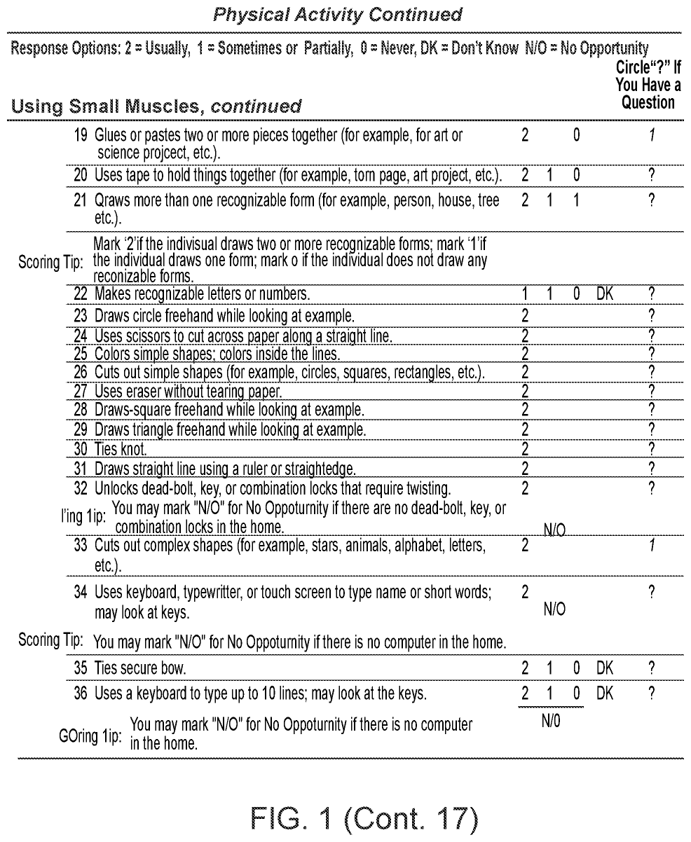

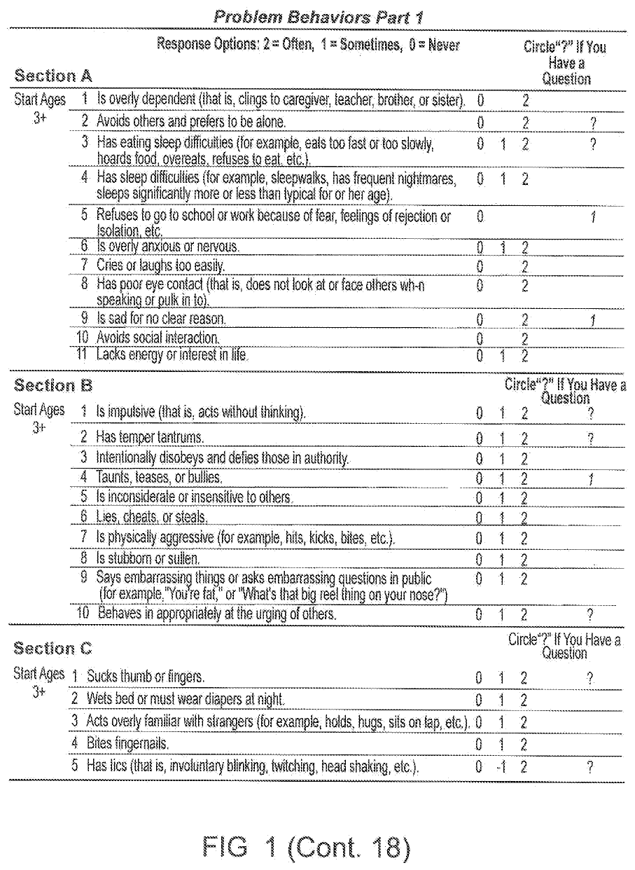

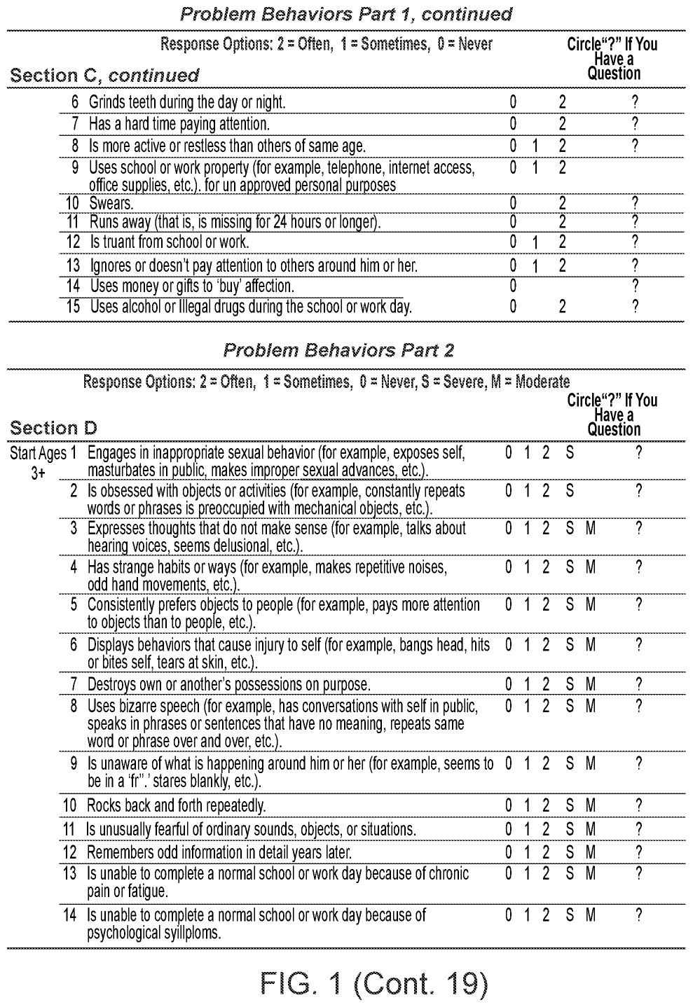

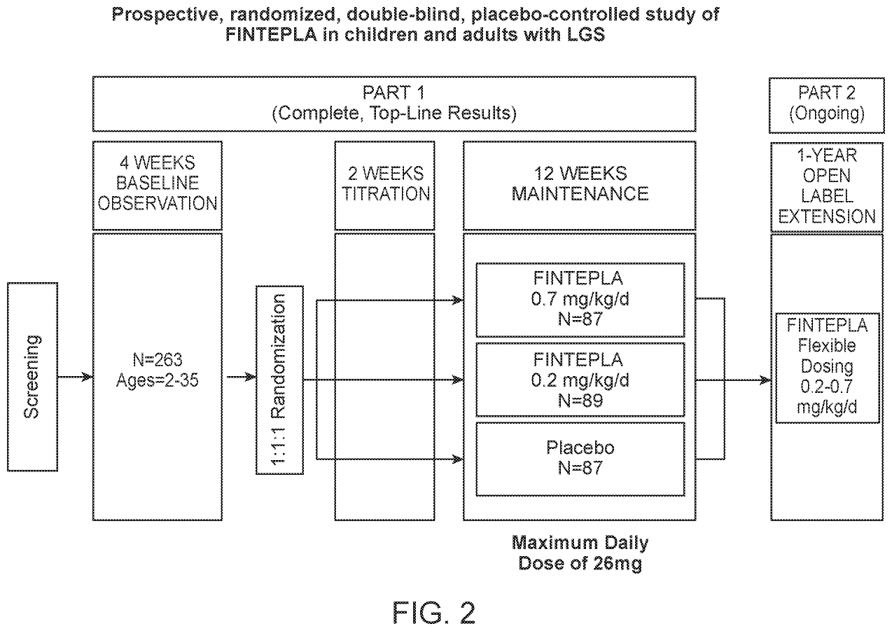

An exemplary VABS assessment is shown in FIG. 1. Some VABS assessments assess intellectual disability (ID), pervasive developmental disorders (PDD), and specific developmental disorders (SDD). “Intellectual disability” is sometimes also referred to as a “general learning disability” or “mental retardation” and is sometimes defined as having an intelligence quotient (IQ) under 70. The term “intelligence quotient” refers to a measure of human intelligence wherein raw scores are adjusted so that the median person has an IQ of 100 and the standard deviation is 15. PDD sometimes involve a delay in the development of basic functions such as socialization and communication. Exemplary PDD include autism, Asberger syndrome, childhood disintegrative disorder, and Rett syndrome. As used herein, SDD refers to delays in development of one specific area of basic function, such as delay in speech articulation (i.e., speech impairment) or specific disorder of arithmetic skills (dyscalculia), as oppose to more generalized delays in development of PDD.

The diagnosing can also involve a clinician's impression of symptoms. As used herein, the term “clinician” includes not only medical and psychological professions such as physicians, psychologists, psychiatrists, and nurses, but also non-professionals who observe the patient, such as a family member who resides with the patient. In some cases, the clinician is a medical or psychological professional. The term “impression of symptoms” does not include EEG measurements, the frequency of seizures, or the type of seizures, which are discussed separately. Instead, the “impression of symptoms” includes, for example, the clinician's subjective assessment of whether the overall condition of the patient has improved, worsened, or remained constant. In some cases the clinician's impression of symptoms is collected using a Clinical Global Impression (CGI) rating scale, which can include the section about severity of illness (CGI-S), global improvement (CGI-I), and efficacy index (CGI-E). For example, a CGI might ask the clinician to rate the patient's severity of symptoms on a scale: 1 (not at all ill), 2 (borderline ill), 3 (mildly ill), 4 (moderately ill), 5 (extremely ill). By comparing the numerical responses over time, an assessment of the effect of the administering can be determined.

The diagnosing can involve recording an electroencephalogram (EEG) measurement. In some cases the measurement is an awake EEG. In some cases the EEG shows an interictal showing of slow spike-wave complexes and fast activity during sleep. The term “interictal” refers to measurements between seizures. In some cases the awake EEG measurement shows a slow spike wave of less than 3 Hz, a spike of less than 70 ms, a sharp wave of 70 ms to 200 ms, and a positive deep trough following the sharp wave, a negative wave of 350 ms to 400 ms following the positive deep trough, or a combination thereof. In some cases, the awake EEG shows all of the elements recited in the previous sentence. In The term “spike-and-wave” refers to an EEG signal that is typically observed during an epileptic seizure and can be regular and symmetrical.

The diagnosing can involve assessing an MRI image of the patient's brain. In some cases the diagnosing is based on: (i) specific finding on EEG of interictal showing of slow spike-wave complexes and fast activity during sleep; (ii) the presence of tonic seizures; and (iii) an MRI compatible with LGS.

In addition, the method can involve monitoring an effect of the administration selected from the group consisting of: seizure frequency, cognitive function, clinician's impression of symptoms, and electroencephalogram (EEG) measurement.

For example, the monitoring can employ the same assessments and steps described above regarding the diagnosing step. For instance, the monitoring can include assessing the patient in one or more of the eight aspects of executive mentioned above regarding BRIEF, i.e. inhibition, shifting attention, emotional control, initiation, working memory, planning, organization, and self-monitoring, e.g. using a BRIEF analysis. The monitoring can also assess the intensity of any intellectual disabilities (ID), pervasive developmental disorders (PDD), specific developmental disorders (SDD), or combinations thereof, e.g. using a VABS assessment. The monitoring can also include clinician's impression of symptoms, e.g. using a CGI assessment. The monitoring can include an EEG measurement, e.g. as described regarding the diagnosing step.

In some cases, the monitoring step indicates an improvement in the patient. The indication of improvement in the patient can be based on one or more measurements, wherein if the indication of improvement is based on two or more measurements, the time interval between the measurements can be 1 day or more, such as 10 days or more, 30 days or more, or 90 days or more. For example, the indication of improvement can be based on three assessments conducted at day 0, day 5, and day 20, which corresponds to an indication based on three measurements performed over a time interval of 10 days or more. The monitoring can show an improvement due to a decrease in seizure frequency. The monitoring of can show an improvement in cognitive function as a result of the administering, e.g. an improvement in executive function or a reduction in ID, PD, SDD, or a combination thereof. These improvements can be shown by, for example, a BRIEF or VABS assessment. In some cases, a clinician's impression of symptoms shows a reduction in symptoms of LGS. For instance, the clinician's impression of symptoms can be assessed using a CGI assessment. In some cases, an EEG measurement shows an improvement in the patient's condition, e.g. a reduction in signals, i.e. brain waves, associated with LGS. In some cases, the indication of improvement of the patient is based on one or more of: seizure frequency, cognitive function, clinician's impression of symptoms, and EEG measurement, such as two or more of such parameters, three or more of such parameters, or four or more of such parameters.

The invention is further illustrated in the following Example.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Study Overview

A randomized, double-blind, placebo-controlled trial was conducted to assess the effect of administering fenfluramine hydrochloride as an adjunctive therapy as an oral solution to children and adults with Lennox-Gastaut syndrome (LGS). The study assessed the effect of such administration on seizures experienced by the subjects.

The fenfluramine hydrochloride was supplied as an oral solution in concentrations of 1.25, 2.5, and 5 mg/mL. Subjects were randomized to receive 1 of 2 doses of fenfluramine hydrochloride (0.2 mg/kg/day, 0.8 mg/kg/day; maximum dose: 30 mg/day) or placebo. Study medication were administered twice a day (BID) in equally divided doses. (Note that subjects taking concomitant stiripentol [STP] were randomized to 0.5 mg/kg/day, maximum dose 20 mg/day, or equivalent volume of placebo. Subjects randomized to 0.5 mg/kg/day were included in analyses with the subjects randomized to 0.8 mg/kg/day not taking STP.)

Note that a daily dose of 0.20 mg/kg/day of fenfluramine hydrochloride corresponds approximately to 0.17 mg/kg/day of fenfluramine free base.

The parents/caregivers used a diary every day to record the number of seizures, type of seizures, time and duration of seizures, whether the seizure resulted in a drop, dosing of study drug, and use of rescue medication. Seizures that result in a drop are defined as seizures involving the entire body, trunk, or head that led to a fall, injury, slumping in a chair, or the subject's head hitting a surface or that could have led to a fall or injury, depending on the patient's position at the time of the seizure.

The cohort included 263 randomized subjects from the United States, Canada, Europe, and Australia. 87 were randomized into 0.7 mg/kg/day of fenfluramine hydrochloride, 89 were randomized into 0.2 mg/kg/day of fenfluramine hydrochloride, and 87 were randomized into a placebo.

### Candidate Inclusion Criteria

Subject is male or nonpregnant, nonlactating female, age 2 to 35 years, inclusive as of the day of the Screening Visit. Female subjects of childbearing potential must not be pregnant or breast-feeding. Female subjects of childbearing potential must have a negative urine or serum pregnancy test at screening. Subjects of childbearing or child-fathering potential must be willing to use medically acceptable forms of birth control, which includes abstinence, while being treated on this study and for 90 days after the last dose of study drug.

Subject must have a diagnosis of Lennox-Gastaut syndrome, where seizures that result in drops are not completely controlled by current antiepileptic treatments. (Subjects without a formal diagnosis may still be enrolled after review and consultation between the Investigator and Sponsor, and in some cases, the Epilepsy Study Consortium. Final decisions on enrollment are at the discretion of the sponsor if all other criteria are met.)

Subjects must meet all of the following 4 criteria for Lennox-Gastaut syndrome, as defined in this protocol:

Onset of seizures at 11 years of age or younger.

Multiple seizure types (must include TS or TA), including countable motor seizures that result in drops. Countable motor seizure types eligible for inclusion are: GTC, TS, CS, AS, FS with observable motor symptoms, and MS with a drop.

Abnormal cognitive development.

Evidence of EEG in the medical history that shows abnormal background activity accompanied by interictal bilaterally synchronous slow spike and wave pattern <2.5 Hz. (Acceptable evidence includes a copy of the EEG trace, EEG report, or physician note that appropriately describes the EEG findings.)

Subject must have had at least 8 drop seizures in the last 4 weeks prior to Screening (minimum of 4 drop seizures in the first two weeks and 4 in the last two weeks before Screening), by parent/guardian report to Investigator or investigator medical notes

Receiving at least 1 concomitant AED and up to 4 concomitant AEDs, inclusive. KD and VNS are permitted but do not count towards the total number of AEDs. Rescue medications for seizures are not counted towards the total number of AEDs.

All medications or interventions for epilepsy (including KD and VNS) must be stable for at least 4 weeks prior to screening and are expected to remain stable throughout the study.

Subject has been informed of the nature of the study and informed consent has been obtained from the legally responsible parent/guardian.

Subject has provided assent in accordance with Institutional Review Board (IRB)/Ethics Committee requirements, if capable.

Subject's parent/caregiver is willing and able to be compliant with diary completion, visit schedule and study drug accountability.

### Candidate Exclusion Criteria

Subject has a known hypersensitivity to fenfluramine or any of the excipients in the study medication.

Subject's etiology of seizures is a degenerative neurological disease.

Subject has a history of hemiclonic seizures in the first year of life.

Subject only has drop seizures in clusters, where individual seizures cannot be counted reliably.

Subject has pulmonary arterial hypertension.

Subject has current or past history of cardiovascular or cerebrovascular disease, such as cardiac valvulopathy, myocardial infarction or stroke, or clinically significant structural cardiac abnormality, including but not limited to mitral valve prolapse, atrial or ventricular septal defects, patent ductus arteriosus (note: Patent Foramen Ovale or a bicuspid valve are not considered exclusionary).

Subject has current or recent history of Anorexia Nervosa, bulimia, or depression within the prior year that required medical treatment or psychological treatment for a duration greater than 1 month.

Subject has a current or past history of glaucoma.

Subject has had an anoxic episode requiring resuscitation within 6 months of the Screening Visit.

Subject has moderate or severe hepatic impairment. Asymptomatic subjects with mild hepatic impairment (elevated liver enzymes <3×ULN and/or elevated bilirubin <2×ULN) may be entered into the study after review and approval by the Medical Monitor in conjunction with the Sponsor, in consideration of comorbidities and concomitant medications.

Subject has severe renal impairment (estimated glomerular filtration rate <30 mL/min/1.73 m²)

Subject is receiving concomitant therapy with any of the following: centrally-acting anorectic agents; monoamine-oxidase inhibitors; any centrally-acting compound with clinically appreciable amount of serotonin agonist or antagonist properties, including serotonin reuptake inhibition; other centrally-acting noradrenergic agonists, including atomoxetine; or cyproheptadine. (Note: Short-term medication requirements for prohibited medications will be handled on a per case basis by the Medical Monitor.)

Subject has positive result on urine or serum tetrahydrocannabinol (THC) Panel or whole blood cannabidiol (CBD) at the Screening Visit.

Subject is taking felbamate for less than 1 year prior to screening and/or does not have stable liver function and hematology laboratory tests, and/or the dose has not been stable for at least 60 days prior to the Screening Visit.

Subject is known to be human immunodeficiency virus (HIV) positive.

Subject is known to have active viral hepatitis (B or C)

Subject is currently receiving an investigational product.

Subject has participated in another clinical trial within the past 30 days (calculated from that study's last scheduled visit). Participation in nontreatment trials will be reviewed by the medical monitor.

Subject is at imminent risk of self-harm or harm to others, in the Investigator's opinion, based on clinical interview and responses provided on the Columbia-Suicide Severity Rating Scale (C-SSRS). Subjects must be excluded if they report suicidal behavior in the past 6 months as measured by the C-SSRS at Screening or Baseline, which includes suicidal ideation with intent and plan. If a subject reports suicidal ideation on Item 4 without specific plan, and the Investigator feels that the subject is appropriate for the study considering the potential risks, the Investigator must document appropriateness for inclusion, and discuss with the parent/caregiver to be alert to mood or behavioral changes, especially around times of dose adjustment.

Subject is unwilling or unable to comply with scheduled visits, drug administration plan, laboratory tests, other study procedures, and study restrictions.

Subject is institutionalized in a general nursing home (ie, in a facility that does not provide skilled epilepsy care).

Subject does not have a reliable caregiver who can provide seizure diary information throughout the study.

Subject has a clinically significant condition, including chronic obstructive pulmonary disease, interstitial lung disease, or portal hypertension, or has had clinically relevant symptoms or a clinically significant illness in the 4 weeks prior to the Screening Visit, other than epilepsy, that would negatively impact study participation, collection of study data, or pose a risk to the subject.

### Procedure

The administration was conducted for 20 weeks. There was a 4-week baseline, a 2-week titration, a 12-week maintenance, and a 2-week taper or transition period.

The 4-week Baseline Period consisted of the establishment of initial eligibility during a screening visit to include an assessment of cardiac parameters (ECG and ECHO), followed by an observation period where subjects were assessed for baseline seizure frequency based on recordings of daily seizure activity entered into a diary. Upon completion of the Baseline Period, subjects who qualified for the study were randomized (1:1:1) in a double-blind manner to receive 1 of 2 doses of fenfluramine hydrochloride (0.2 mg/kg/day, 0.8 mg/kg/day; maximum dose: 30 mg/day [or 0.5 mg/kg/day, maximum 20 mg/day, for subjects taking concomitant STP]) or placebo. Randomization was stratified by weight (<37.5 kilograms [kg], ≥37.5 kg) to ensure balance across treatment arms, and at least 25% of subjects will be in each weight group. All subjects were titrated to their blinded randomized dose over a 2-week Titration Period. Following titration, subjects continued treatment at their randomly assigned dose over a 12-week Maintenance Period. Total treatment time from the beginning of the Titration Period through the end of the Maintenance Period is 14 weeks. Subjects had ECG and ECHO assessments at weeks 6 and 14 during the Maintenance Period. At the end of the Maintenance Period (or early discontinuation), all subjects underwent a blinded 2-week taper or transition period (Post-Dosing Follow-Up) depending on whether they exit the study or are enrolled in Part 2, the long-term open-label extension, respectively.

Change in seizure frequency was assessed as the drops between baseline and the combined Titration and Maintenance Periods (T+M). Also assessed was the proportion of subjects who achieve a ≥50% reduction from baseline in the frequency of seizures that result in drops. Also assessed was changes in the Clinical Global Impression—Improvement rating, as assessed by the Principal Investigator.

The following parameters were assessed: adverse events (AEs), laboratory parameters, physical examination, neurological examination, vital signs (blood pressure, heart rate, temperature, and respiratory rate), electrocardiograms (ECG), echocardiograms (ECHO), body weight, BMI, and Behavior Rating Inventory of Executive Function (BRIEF).

### Behavior Rating Inventory of Executive Function (BRIEF)

The Behavior Rating Inventory of Executive Function (BRIEF) is a parent or teacher report measure designed to address the multidimensional nature of the executive function construct and will be used to assess the effects of fenfluramine on cognition. The BRIEF assesses eight theoretically and statistically derived subdomains of executive function. It was designed to be used for a wide range of childhood disorders in order to augment traditional clinic-based assessments, and to provide an increased level of ecological validity for clinical assessments. Age-appropriate versions of the BRIEF (BRIEF-P: 2- to 5-year-olds; BRIEF: 6- to 18-year-olds; BRIEF-A: 19- to 35-year-olds), were conducted.

### Vineland Adaptive Behavior Scale (VABS)

Subjects were also assessed using the Vineland Adaptive Behavior Scale (VABS), as shown in FIG. 1.

Results

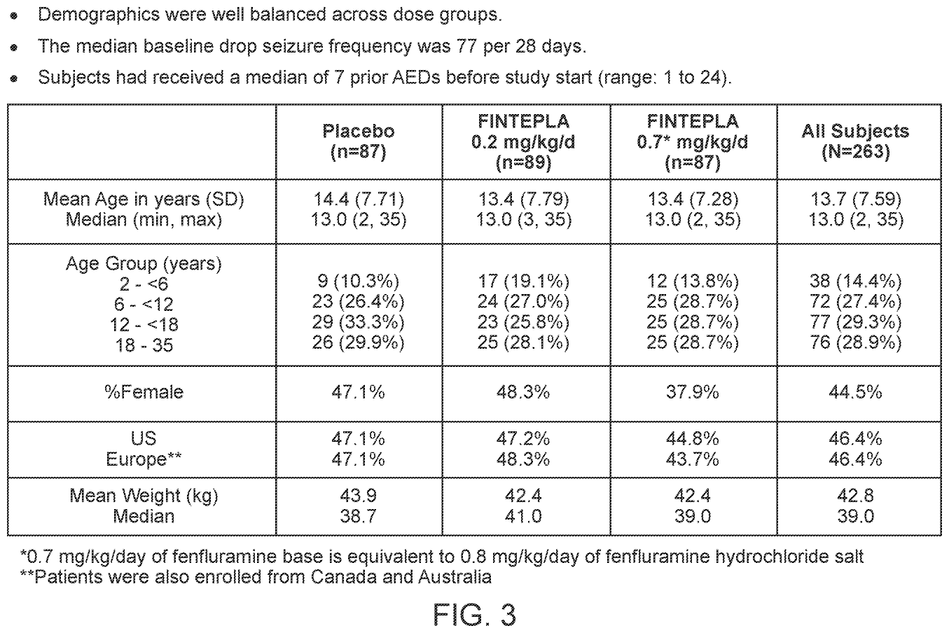

FIG. 3 shows the age, gender, and weight of the subjects.

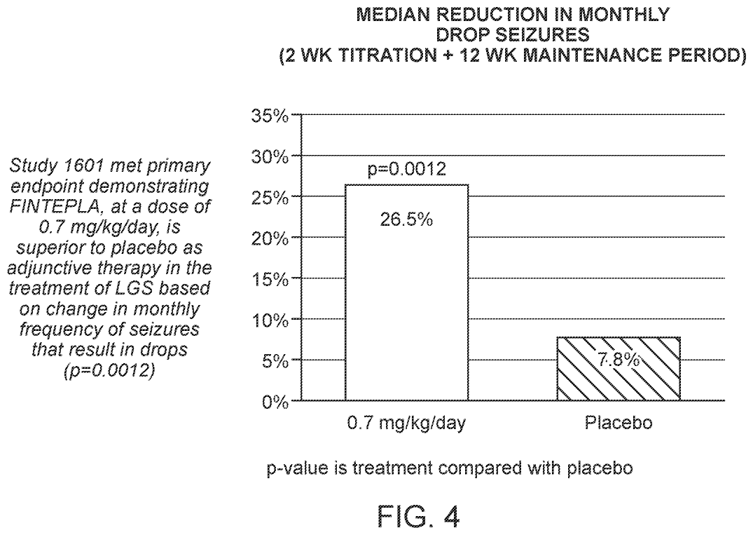

As shown in FIG. 4, the median reduction in monthly drop seizures for subjects receiving 0.7 mg/kg/day of fenfluramine HCl was 26.5%, compared to only 7.8% with placebo. This difference corresponds to a p-value of 0.0012, strongly suggesting a statistically significant effect of the fenfluramine administration.

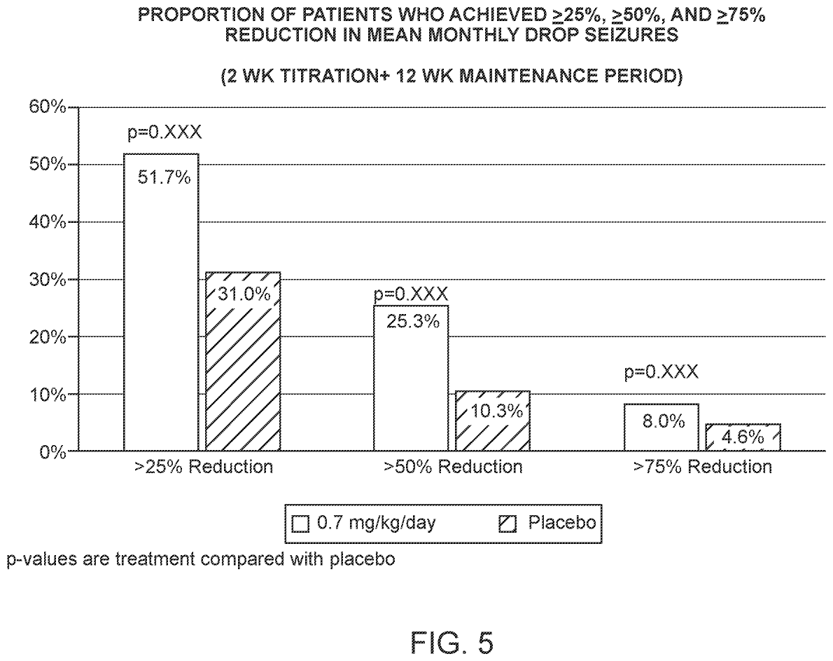

FIG. 5 shows the proportions of patients who experienced each category of reductions in monthly drop seizures. This relates to 0.7 mg/kg/day of fenfluramine HCl versus placebo. For example, 51.7% of the 0.7 mg/kg/day subjects experienced a reduction of 25% or more, whereas only 31% of such placebo patients experienced such a reduction.

FIG. 5 shows the proportions of subjects with particular reductions in mean monthly drop seizures, for 0.7 mg/kg/day fenfluramine HCl. For example, a 25% or more reduction in drop seizures were observed for 51.7% of the 0.7 mg/kg/day patients compared to 31% for placebo patients.

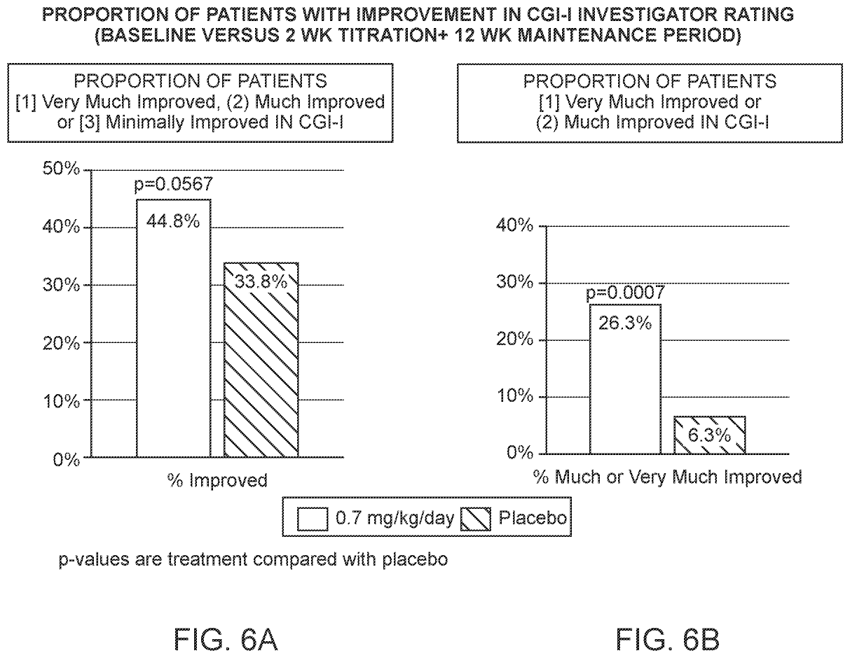

FIGS. 6A and 6B shows the proportion of patients with improvements in the CGI-I Investigator Rating. For example, 26.3% of the 0.7 mg/kg/day of fenfluramine HCl subjects were classified as “much or very much improved”, compared to only 6.3% of the placebo subjects. This corresponds to a p-value of 0.0007, highly suggesting a positive effect in CGI-I from administration of fenfluramine.

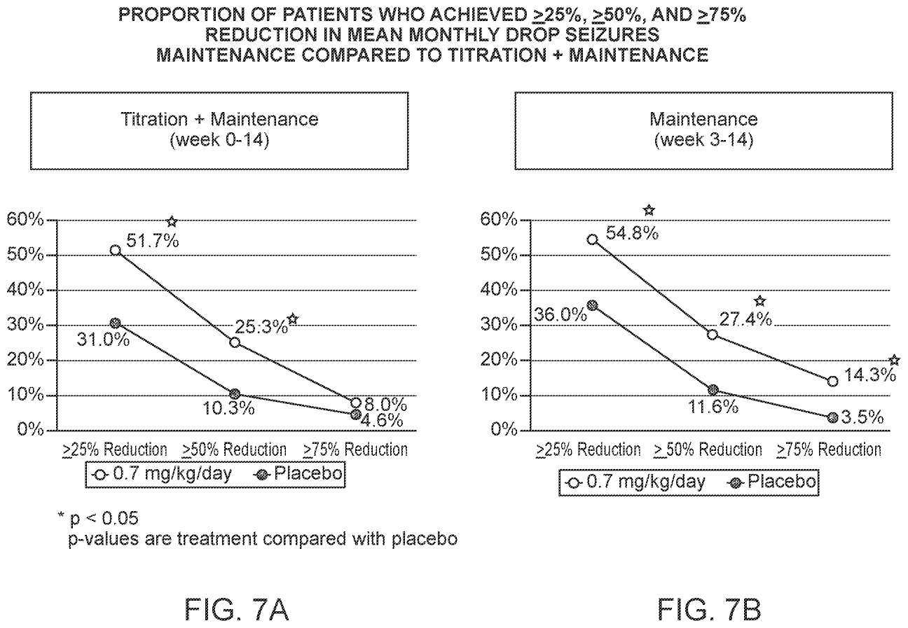

FIGS. 7A and 7B shows the proportion of subjects who achieved certain amounts of reduction in monthly drop seizures. The data relates to subjects with 0.7 mg/kg/day of fenfluramine HCl versus placebo. For example, as shown on the left graph, for the titration plus maintenance period (week 0-14), a 51.7% reduction was seen for the 0.7 mg/kg/day group, compared to only a 31% reduction for the placebo group.

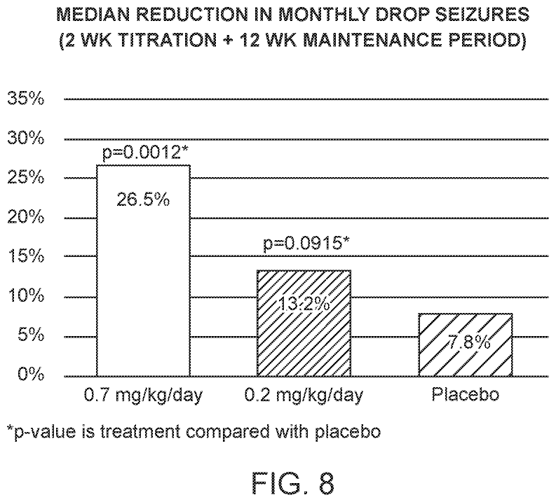

FIG. 8 shows medium reduction in monthly drop seizures for the time period of 2 week titration plus 12 week maintenance period. In particular, compared to placebo, the 0.7 mg/kg/day fenfluramine HCl group had a 26.5% reduction versus 7.8% reduction, corresponding to a p-value of 0.0012. For the 0.2 mg/kg/day fenfluramine HCl group, the comparison was 13.2% to 7.8%, for a p-value of 0.0915.

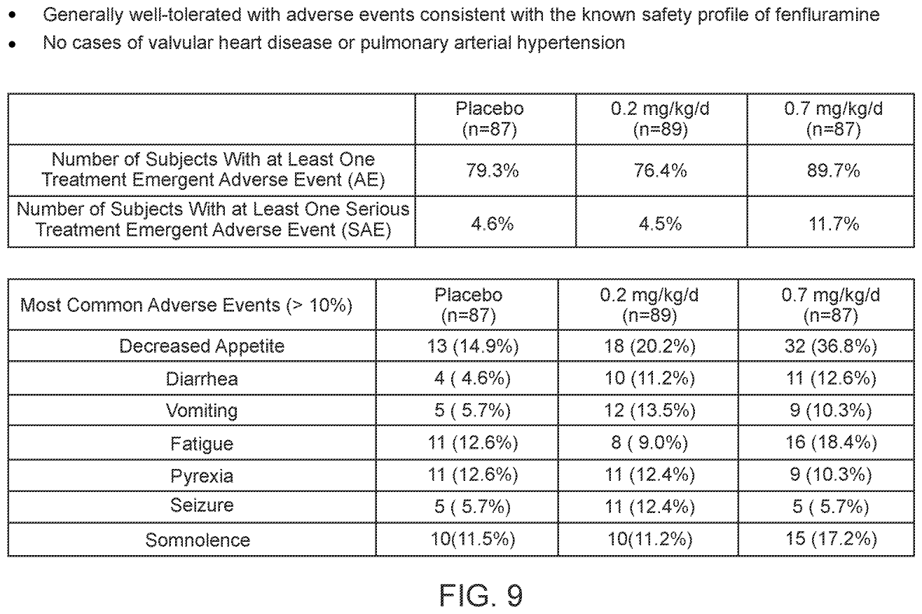

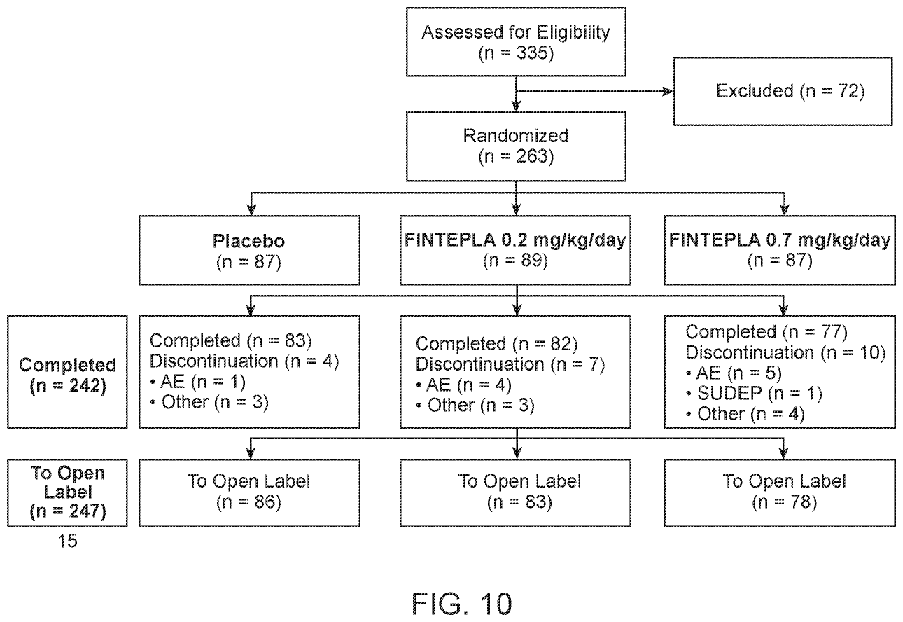

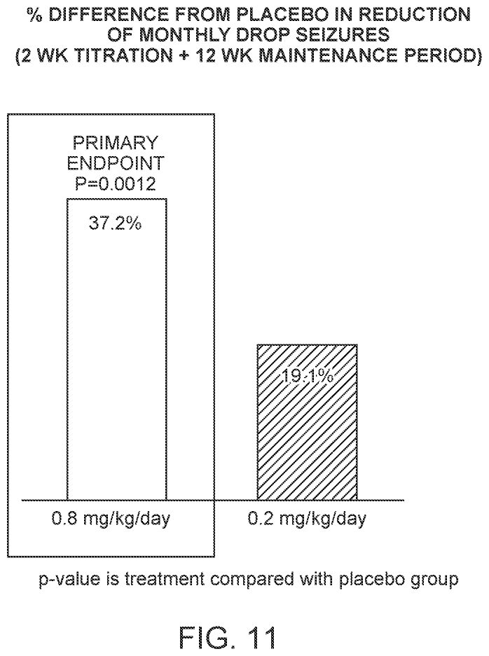

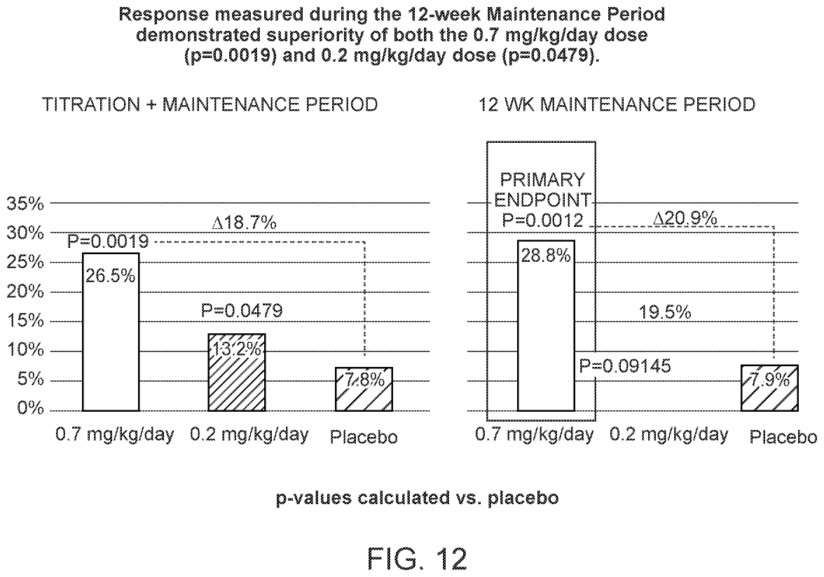

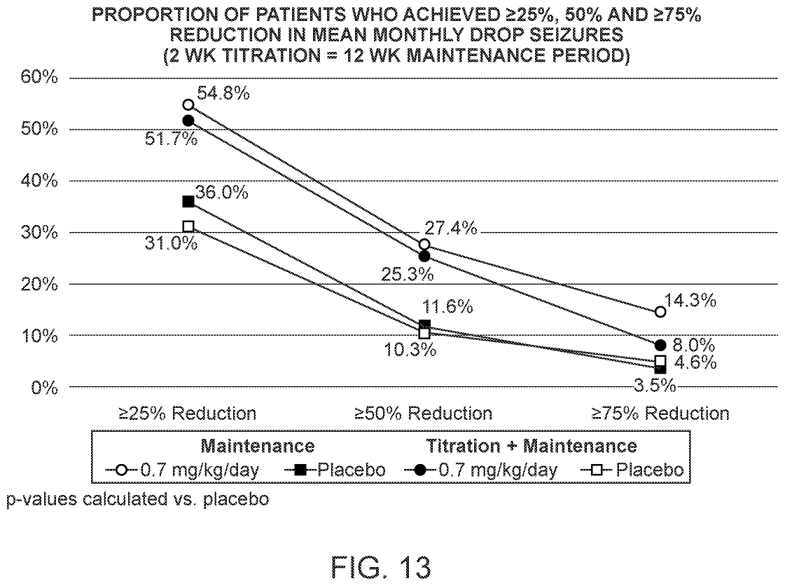

FIG. 9 shows safety data from the study. The administrations were generally well tolerated was adverse events consistent with the known safety profile of fenfluramine No cases of valvular heart disease or pulmonary arterial hypertension were observed. For instance, the 0.7 mg/kg/day group had 11.7% with at least one serious treatment emergent adverse event (SAE), compared to 4.6% with placebo.

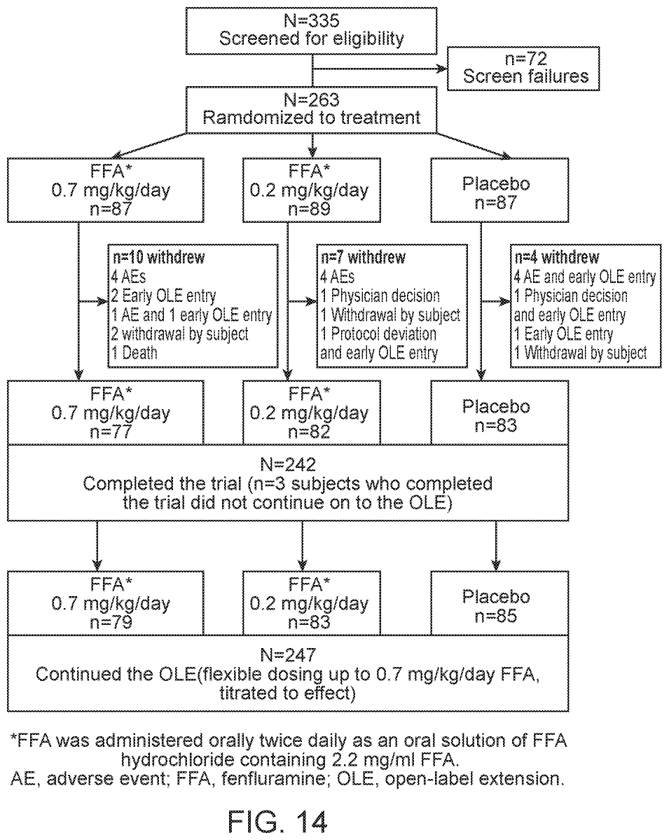

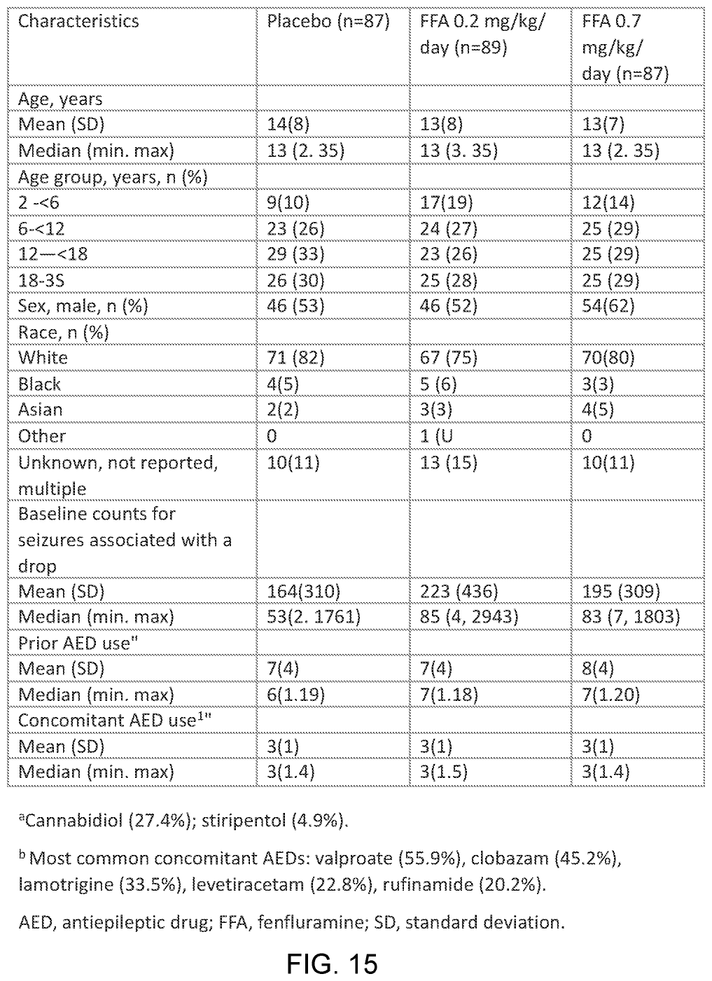

FIGS. 14 and 15 show additional demographic data about the study participants.

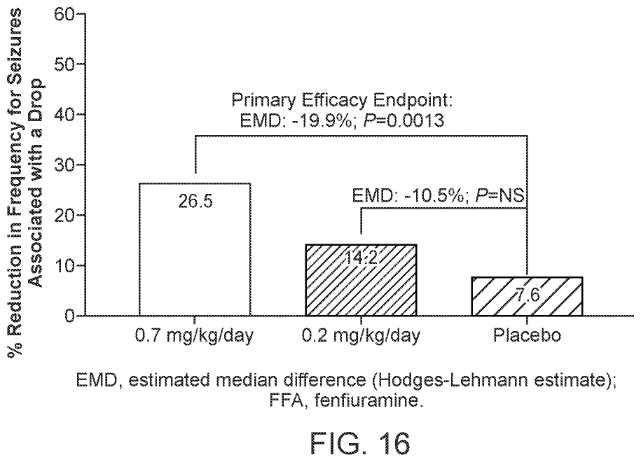

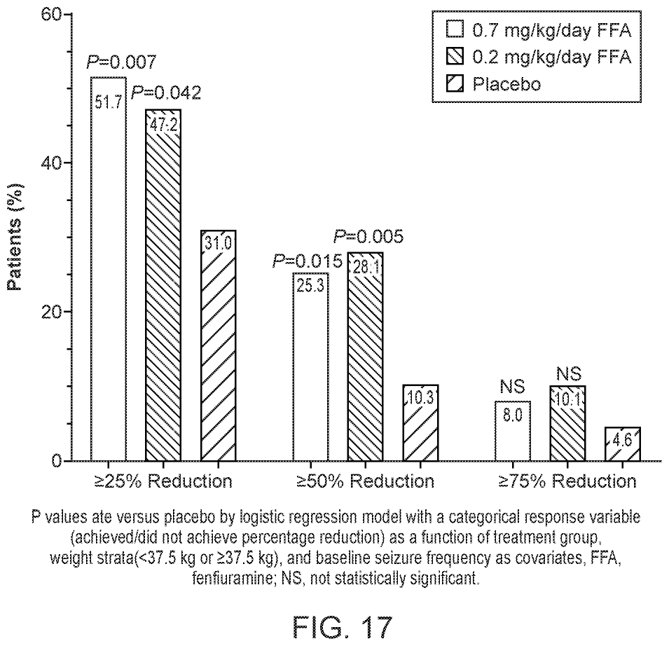

FIGS. 16 and 17 show the primary efficacy endpoints: estimated median difference from placebo, using the Hodges-Lehmann Estimate. In particular, FIG. 16 shows the reduction in seizures associated with a drop, i.e. associated with a fall. In particular, the 0.7 mg/kg/day group experienced 26.5% reduction in drop seizures, whereas this value was 14.2% and 7.6% for 0.2 mg/kg/day and placebo. FIG. 17 shows the percentage of patients who experienced at least 25%, 50%, or 75% reduction in drop seizures. For example, the number of patients who experienced at least 25% reduction in seizures was 51.7% for the 0.7 mg/kg/day group, which corresponds to a p value of 0.007 in relation to the value of 31.0% for the placebo group.

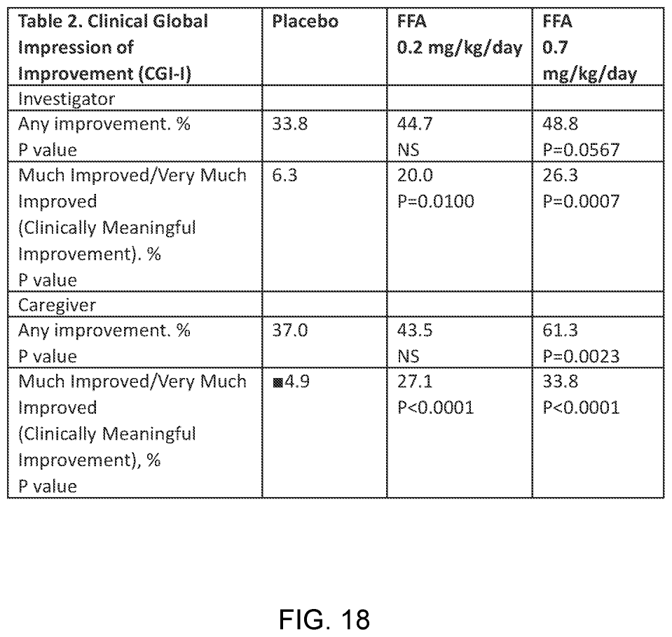

FIG. 18 describes for each group the Clinical Global Impression of Improvement (CGI-I). For instance, the percentage of patients who were “much improved” or “very much improved” was 26.3% for the 0.7 mg/kg/day group, corresponding to a p-value of 0.0007 compared with the value of 6.3% for the placebo group.

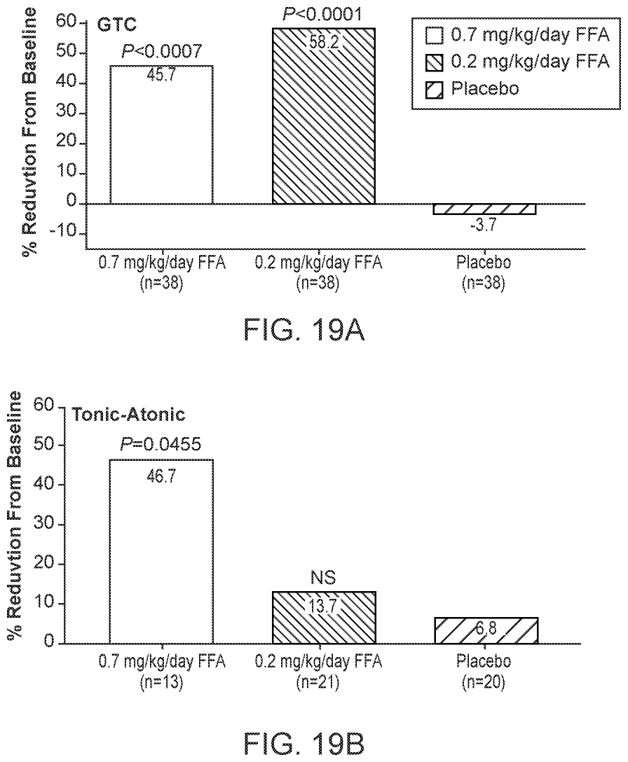

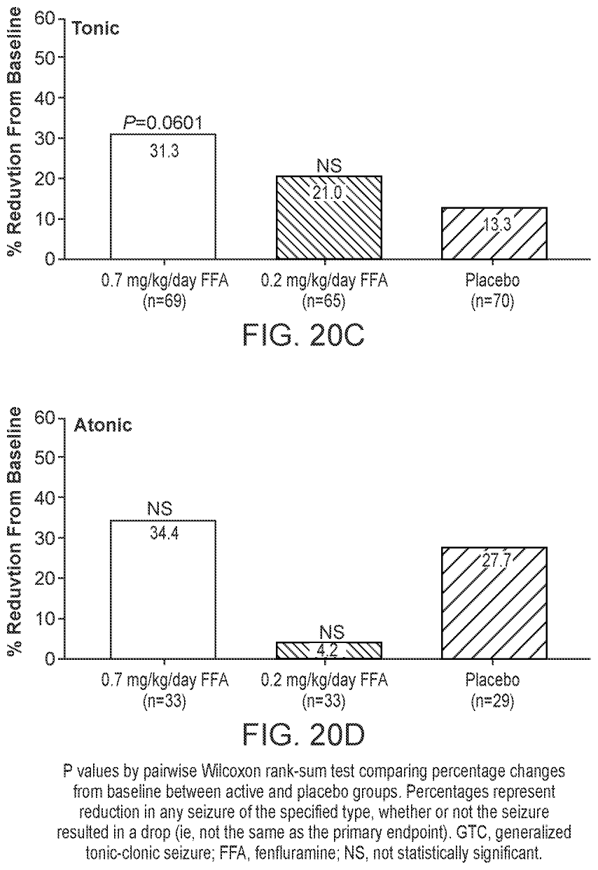

FIGS. 19 and 20 show reduction from baseline of seizures for seizure types general tonic-clonic (FIG. 19, panel A), tonic-atonic (FIG. 19, panel B), tonic (FIG. 20, panel C), and atonic (FIG. 20, panel D). For GTC, the placebo group experienced a reduction of negative 3.7%, whereas this value was 58.2% or 45.7% for 0.2 and 0.7 mg/kg/day groups. The associated p-values were less than 0.001, strongly suggesting a beneficial pharmaceutical effect on incidence of GTC. Similarly, a statistically significant reduction in tonic-atonic seizures was noted for patients administered 0.7 mg/kg/day. In particular, the 0.7 mg/kg/day group experienced a 46.7% reduction compared with a 6.8% reduction in the placebo group, and the associated p-value was 0.0455. As shown in panel C of FIG. 20, administration of 0.7 mg/kg/day reduced tonic seizures by 31.3%, compared with 13.3% for the placebo group, and a p-value of 0.0601 was found.

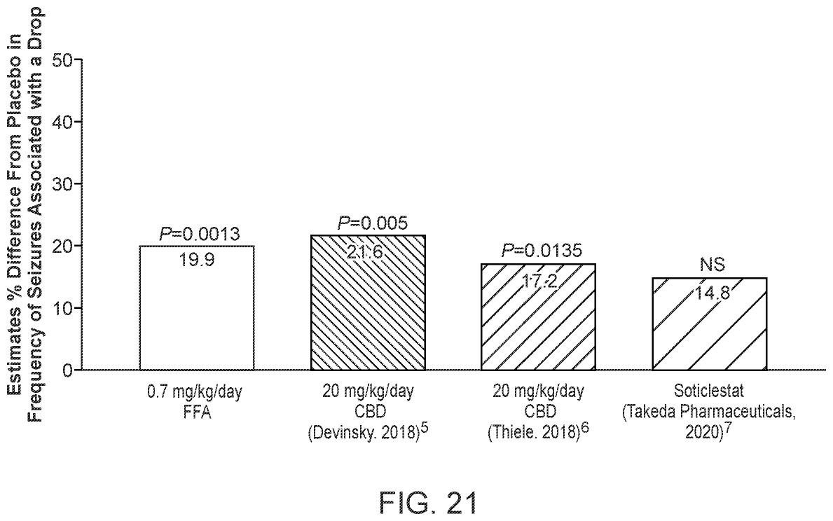

FIG. 21 shows a comparison of reduction in seizures associated with drops by administration of fenfluramine compared to administration of other pharmaceutical drugs by other researchers. In particular, administering 0.7 mg/kg/day resulted in a 19.9% reduction, with a p-value of 0.0013. As reported by Devinksy in 2018 and Thiele in 2018, administering 20 mg/kg/day of cannabidiol (CBD) resulted in either 21.6% or 17.2% reductions, corresponding to p-values of 0.005 and 0.0135, respectively. In 2020, Takeda Pharmaceuticals reported that administration of Soticlestat resulted in 14.8% reduction in drop seizures, but this result was not found to be statistically significant.

### Statistical Methods

The primary analyses of the study will be performed on data from after the last subject enrolled in the cohort has completed the last study visit of Part 1. Subjects randomized to 0.5 mg/kg/day (ie, those taking concomitant STP) will be grouped with subjects randomized to 0.8 mg/kg/day for all efficacy analyses.

Primary Efficacy Analysis: The primary efficacy endpoint for Part 1 is the percent change in frequency of seizures that result in drops (DSF: drop seizure frequency) per 28 days between the T+M and Baseline periods in Cohort A. The DSF will be calculated from all available data collected during the Baseline and treatment periods without imputation. The percent change in DSF will be calculated as the change in DSF between T+M and Baseline/DSF during Baseline 100. Both the mean and median percent change in DSF will be presented. The primary endpoint will be assessed using a non-parametric rank analysis of covariance (ANCOVA) with treatment and weight group as factors, rank baseline DSF as a covariate, and rank percent change in DSF from baseline as the response variable. The primary analysis will compare the ZX008 (development name for fenfluramine) 0.8 mg/kg/day group to the placebo group at the alpha=0.05 level of significance. The difference between the ZX008 0.8 mg/kg/day and placebo groups in percent change in DSF, and its 95% confidence interval, will be estimated using the Hodges-Lehmann method. As a sensitivity analysis, the primary endpoint will also be analyzed using a parametric ANCOVA that incorporates treatment group and weight group as factors, log baseline DSF as a covariate; and log DSF during T+M as the response variable. Another sensitivity analysis will use a Wilcoxon rank-sum test to compare the ZX008 0.8 mg/kg/day group to the placebo group. Part 1 Cohort B will be analyzed using analogous methods.

A key secondary analysis will compare the ZX008 0.2 mg/kg/day group to the placebo group on percent change in DSF using the same method as the primary analysis. Other key secondary analyses will compare the ZX008 0.8 and 0.2 mg/kg/day groups to the placebo group on the proportion of subjects who achieve a ≥50% reduction from baseline in DSF. The analyses will utilize independent logistic regression models that incorporate the same factors and covariate as the primary analysis. Two other key secondary analyses will compare both ZX008 dose groups (independently) to placebo on the proportion of subjects assessed by the Principal Investigator as minimally, much, or very much improved on the Clinical Global Impression—Improvement (CGI-I). The comparisons will employ separate Cochran-Mantel-Haenszel tests (CMH) stratified by weight strata. A serial gatekeeper strategy will be used to maintain the Type 1 error rate at α=0.05 across the family of analyses that support the primary and key secondary objectives.

All safety data will be appropriately analyzed by treatment group. The number and percentage of subjects with AEs will be displayed by body system and preferred term using the Medical Dictionary for Regulatory Activities (MedDRA). Summaries in terms of severity and relationship to study drug will also be provided. Adverse Events of Special Interest (AESI) and Serious AEs (SAEs) will be summarized separately in a similar manner Laboratory tests, vital signs, physical examinations, neurological examinations, ECG, Doppler echocardiogram, chest x-ray (France and Netherlands only), EEG (Italy only), C-SSRS, Tanner Staging results, etc., will be summarized appropriately, by treatment. All safety summaries will be based on the Safety Population.

Model derived plasma PK parameters (Cmax_ss, Cmin_ss, AUC0-t, AUC0-24) will be summarized descriptively by treatment group, when sufficient data are available. A Population pharmacokinetic (PopPK) model of fenfluramine, previously developed using data from healthy adults and pediatric patients with Dravet syndrome, will be updated to include the fenfluramine and norfenfluramine concentration-time data collected during the Maintenance Period of Part 1. This model will be informed by all relevant data available at the time of data collection (both adults and pediatrics). The population mean and interindividual variability estimates from the fit of the PopPK model will be summarized. The results from the PopPK modeling will be reported separately and conducted according to a separate SAP.

The phase 3 study in LGS met its primary endpoint, with fenfluramine 0.7 mg/kg/day demonstrating significant improvement in frequency of seizures associated with a drop (Hodges-Lehmann EMD (estimated median difference)) from placebo of 19.9%.

Fenfluramine was highly effective in reducing generalized tonic-clonic seizures (GTCs) by 46% and 58% in the 0.7 and 0.2 mg/kg/day fenfluramine groups, respectively, compared to worsening of 3.7% in the placebo group. Fenfluramine also demonstrated a 47% reduction in tonic atonic seizures in the 0.7 mg/kg/day group.

Notwithstanding the appended claims, the disclosure is also defined by the following clauses:
1. A method of treating symptoms of Lennox-Gastaut syndrome (LGS) in a patient diagnosed with LGS comprising administering an effective dose of fenfluramine or a pharmaceutically acceptable salt thereof to the patient.
2. The method of clause 1, wherein the effective dose is administered in the form of one or more dosage forms for oral, injectable, transdermal, inhaled, nasal, rectal, vaginal or parenteral delivery.
3. The method of clause 2, wherein the dosage form is an oral solution.
4. The method of any one of clauses 1-3, wherein the fenfluramine or the pharmaceutically acceptable salt thereof is administered two times per day.
5. The method of any one of clauses 1-4, wherein the effective dose ranges from 1.5 mg/kg/day to 0.05 mg/kg/day.
6. The method of any one of clause 5, wherein the effective dose ranges from 1.0 mg/kg/day to 0.1 mg/kg/day.
7. The method of clause 6, wherein the effective dose ranges from 0.85 mg/kg/day to 0.15 mg/kg/day.
8. The method of any one of clauses 1-7, wherein the patient is aged 18 or less.
9. The method of any one of clauses 1-8, wherein the patient weighs 50 kg or less.
10. The method of any one of clauses 1-9, wherein the formulation consists essentially only of fenfluramine as the active ingredient and the patient is dosed in an amount of fenfluramine (free base) of 0.7 mg/kg/day up to a maximum of 26 mg/day.
11. The method of any one of clauses 1-10, wherein the fenfluramine or the pharmaceutically acceptable salt thereof is co-administered with one or more co-therapeutic agents and the patient is dosed in an amount of fenfluramine (free base) of 0.7 mg/kg/day.
12. The method of clause 11, wherein the one or more co-therapeutic agents comprises an anticonvulsant.
13. The method of clause 12, wherein the anticonvulsant is selected from the group consisting of carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, and midazolam or a pharmaceutically acceptable salt thereof.
14. The method of clause 13, wherein the anticonvulsant is stiripentol in combination with valproate and/or clobazam and the dose of fenfluramine (free base) is 0.2 mg/kg/day up to a maximum dose of 17 mg/day.
15. The method of any one of clauses 1-14, wherein the patient, prior to the administering, has experienced one or more seizure types selected from the group consisting of: generalized tonic-clonic, tonic-atonic, tonic, and atonic.
16. The method of clause 15, wherein the patient has experienced a generalized tonic-clonic seizure prior to the administering.
17. The method of clause 15 or 16, wherein the patient has experienced a tonic-atonic seizure prior to the administering.
18. The method of any one of clauses 1-17, wherein the patient experiences a 20% or more reduction in seizure frequency after the administering compared with a period of time before the administering.
19. The method of clause 18, wherein the 20% or more is 40% or more.
20. The method of clause 18 or 19, wherein the reduction in seizure frequency is a reduction in generalized tonic-clonic seizures.
21. The method of clause 18 or 19, wherein the reduction in seizure frequency is a reduction in tonic-atonic seizures.
22. The method of any one of the preceding clauses, further comprising treating, preventing, and/or ameliorating generalized tonic clonic seizures in the patient.
23. The method of any one of the preceding clauses, wherein the administering comprises administering an effective dose of fenfluramine or a pharmaceutically acceptable salt thereof to said patient and the patient is dosed in an amount of fenfluramine (free base) of up to 0.7 mg/kg/day and a maximum of 26 mg/day
24. The method of any one of the preceding clauses, further comprising diagnosing the patient with LGS before the administering.
25. The method of any one of the preceding clauses, wherein the diagnosing comprises determining one or more parameters selected from the group consisting of: age of symptom onset, type or types of seizures, seizure frequency, cognitive function, a clinician's impression of symptoms, electroencephalogram (EEG) measurement, and genetic mutation.
26. The method of any one of the preceding clauses, wherein the diagnosing comprises determining that the age of symptom onset was before the eleventh birthday of the patient.
27. The method of any one of the preceding clauses, wherein the diagnosing comprises determining that the patient has multiple types of seizures.
28. The method of any one of the preceding clauses, wherein the diagnosing comprises determining that seizure frequency is an average of 0.5 or more seizures per day over a period of 14 days or more.
29. The method of any one of the preceding clauses, wherein the diagnosing comprises determining a cognitive function selected from the group consisting of: an executive function, an intellectual disability (ID), a pervasive developmental disorder (PDD), a specific developmental disorder (SDD), or a combination thereof.
30. The method of any one of the preceding clauses, wherein determining the executive function involves a Behavior Rating Inventory of Executive Function (BRIEF) assessment; determining the ID, PDD, or SDD involves a Vineland Adaptive Behavior Scale (VABS) assessment; or a combination thereof.
31. The method of any one of the preceding clauses, wherein the diagnosing comprises determining a clinician's impression of symptoms.
32. The method of any one of the preceding clauses, wherein the diagnosing comprises recording an awake EEG measurement.
33. The method of any one of the preceding clauses, wherein the awake EEG measurement shows a slow spike wave.
34. The method of any one of the preceding clauses, wherein the awake EEG measurement shows a slow spike wave of less than 3 Hz, a spike of less than 70 ms, a sharp wave of 70 ms to 200 ms, and a positive deep trough following the sharp wave, a negative wave of 350 ms to 400 ms following the positive deep trough, or a combination thereof.
35. The method of any one of the preceding clauses, wherein the diagnosing comprises determining that the patient has a genetic mutation in one or more genes selected from the group consisting of: CHD2, GABRB3, ALG13, and SCN2A.
36. The method of any one of the preceding clauses, wherein the diagnosing comprises determining that the patient does not have Doose syndrome, Dravet syndrome, or a combination thereof.
37. The method of any one of the preceding clauses, further comprising monitoring an effect of the administration selected from the group consisting of: seizure frequency, cognitive function, clinician's impression of symptoms, and electroencephalogram (EEG) measurement.
38. The method of any one of the preceding clauses, wherein the monitoring comprises determining that seizure frequency has decreased by 25% or more due to the administration.
39. The method of any one of the preceding clauses, wherein the monitoring comprises determining that seizure frequency has decreased by 50% or more due to the administration.
40. The method of any one of the preceding clauses, wherein determining that seizure frequency has decreased is based on monitoring the patient for 30 days or more.
41. The method of any one of the preceding clauses, wherein the monitoring comprises determining a cognitive function selected from the group consisting of: an executive function, an intellectual disability (ID), a pervasive developmental disorder (PDD), a specific developmental disorder (SDD), or a combination thereof.
42. The method of any one of the preceding clauses, wherein determining the executive function involves a Behavior Rating Inventory of Executive Function (BRIEF) assessment; determining the ID, PDD, or SDD involves a Vineland Adaptive Behavior Scale (VABS) assessment; or a combination thereof.
43. The method of any one of the preceding clauses, wherein the monitoring comprises determining that a clinician's impression of symptoms has improved due to the administration.
44. The method of any one of the preceding clauses, wherein the monitoring comprises determining that a clinician's impression of symptoms has increased due to the administration with an EEG measurement.
45. The method of any one of the preceding clauses, wherein the EEG measurement comprises an assessment of a spike-and-wave pattern of the patient.
46. The method of any one of the preceding clauses, wherein the fenfluramine or the pharmaceutically acceptable salt thereof is fenfluramine HCl.
47. The method of any one of the preceding clauses, further comprises monitoring seizure frequency as an effect of the administration.
48. The method of any one of the preceding clauses, wherein the seizure frequency is a frequency of drop seizures.
49. The method of any one of the preceding clauses, wherein seizure frequency is a frequency of generalized tonic-clonic seizures, tonic-atonic seizures, or a combination thereof.
50. A method of treating, preventing, and/or ameliorating generalized tonic clonic seizures in a patient diagnosed with Lennox-Gastaut syndrome (LGS), comprising:
   administering an effective dose of fenfluramine or a pharmaceutically acceptable salt thereof to said patient and the patient is dosed in an amount of fenfluramine (free base) of up to 0.7 mg/kg/day and a maximum of 26 mg/day.
51. The method of clause 50, wherein the effective dosing is continued on a daily basis over a period of weeks until seizures are reduced by 25% or more.
52. The method of any one of clauses 50-51, wherein the dosage form is an oral solution and dosing is continued on a daily basis over a period of weeks until seizures are reduced by 50% or more.
53. The method of any one of clauses 50-52, wherein the fenfluramine or the pharmaceutically acceptable salt thereof is administered two times per day and dosing is continued on a daily basis over a period of weeks until seizures are reduced by 75% or more.
54. The method of any one of clauses 50-53, wherein the fenfluramine or the pharmaceutically acceptable salt thereof is administered two times per day and dosing is continued on a daily basis over a period of weeks until seizures are reduced by about 46 to 58%.
55. The method of any one of clauses 50-54, wherein the effective dose ranges from 1.5 mg/kg/day to 0.05 mg/kg/day.
56. The method of any one of clauses 50-55, wherein the effective dose ranges from 1.0 mg/kg/day to 0.1 mg/kg/day.
57. The method of any one of clauses 50-56, wherein the effective dose ranges from 0.85 mg/kg/day to 0.15 mg/kg/day.
58. The method of any one of clauses 50-57, wherein the patient is aged 18 or less.
59. The method of any one of clauses 50-58, wherein the patient weighs 50 kg or less.
60. The method of any one of clauses 50-59, wherein the formulation consists essentially only of fenfluramine as the active ingredient.
61. The method of any one of clauses 50-60, wherein the fenfluramine or the pharmaceutically acceptable salt thereof is co-administered with one or more co-therapeutic agents.
62. The method of any one of clauses 50-61, wherein the one or more co-therapeutic agents comprises an anticonvulsant.
63. The method of any one of clauses 50-62, wherein the anticonvulsant is selected from the group consisting of carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, and midazolam or a pharmaceutically acceptable salt thereof.
64. The method of any one of clauses 50-63, wherein the anticonvulsant is stiripentol.
65. A kit for treating symptoms of Lennox-Gastaut syndrome (LGS) in a patient diagnosed with LGS, comprising:
   a formulation comprising a pharmaceutically acceptable carrier and an active ingredient comprising fenfluramine or a pharmaceutically acceptable salt thereof, wherein the formulation is an oral solution;
   instructions for treating the patient diagnosed with LGS by administering the liquid formulation to the patient withdrawn from a container using a pipet.
66. The kit of clause 65, wherein the formulation consists of one milligram of fenfluramine in each milliliter of liquid solution.
67. The kit of clause 65 or 66, wherein the instructions indicate dosing the patient based on patient weight and volume of oral solution administered.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of treating symptoms of Lennox-Gastaut syndrome (LGS) in a patient diagnosed with LGS comprising administering an effective dose of fenfluramine or a pharmaceutically acceptable salt thereof to the patient.

2. The method of claim 1, wherein the effective dose ranges from 1.0 mg/kg/day to 0.1 mg/kg/day and the effective dose is administered as an oral solution.

3. The method of claim 1, wherein the fenfluramine or the pharmaceutically acceptable salt thereof is fenfluramine HCl.

4. The method of claim 1, wherein the fenfluramine or the pharmaceutically acceptable salt thereof is co-administered with one or more anticonvulsants.

5. The method of claim 1, further comprising diagnosing the patient with LGS before the administering by determining one or more parameters selected from the group consisting of: age of symptom onset, type or types of seizures, seizure frequency, cognitive function, a clinician's impression of symptoms, electroencephalogram (EEG) measurement, and genetic mutation.

6. The method of claim 5, wherein the diagnosing comprises determining a cognitive function selected from the group consisting of: an executive function, an intellectual disability (ID), a pervasive developmental disorder (PDD), a specific developmental disorder (SDD), or a combination thereof.

7. The method of claim 6, wherein determining the executive function involves a Behavior Rating Inventory of Executive Function (BRIEF) assessment; determining the ID, PDD, or SDD involves a Vineland Adaptive Behavior Scale (VABS) assessment; or a combination thereof.

8. The method of claim 5, wherein the diagnosing comprises determining a clinician's impression of symptoms.

9. The method of claim 5, wherein the diagnosing comprises recording an awake EEG measurement.

10. The method of claim 9, wherein the awake EEG measurement shows a slow spike wave.

11. The method of claim 10, wherein the awake EEG measurement shows a slow spike wave of less than 3 Hz, a spike of less than 70 ms, a sharp wave of 70 ms to 200 ms, and a positive deep trough following the sharp wave, a negative wave of 350 ms to 400 ms following the positive deep trough, or a combination thereof.

12. The method of claim 5, wherein the diagnosing comprises determining that the patient does not have Doose syndrome or Dravet syndrome.

13. The method of claim 1, further comprising monitoring an effect of the administration selected from the group consisting of: seizure frequency, cognitive function, a clinician's impression of symptoms, and electroencephalogram (EEG) measurement.

14. The method of claim 13, wherein the monitoring comprises determining that seizure frequency has decreased by 25% or more due to the administration based on monitoring the patient for 30 days or more.

15. The method of claim 14, wherein the decrease in seizure frequency is a decrease in drop seizures.

16. The method of claim 14, wherein the decrease in seizure frequency is a decrease in generalized tonic-clonic seizures, a decrease in tonic-atonic seizures, or a combination thereof.

17. The method of claim 13, wherein the monitoring comprises determining a cognitive function selected from the group consisting of: an executive function, an intellectual disability (ID), a pervasive developmental disorder (PDD), a specific developmental disorder (SDD), or a combination thereof.
